(19) United States
(12) Patent Application Publication
(10) Pub. No.: US 2026/0226420 A1
(43) Pub. Date: Aug. 6, 2026

(54) METHODS FOR THE PRODUCTION OF HEPATIC STELLATE CELLS FROM HUMAN PLURIPOTENT STEM CELLS

(71) Applicant: FUJIFILM Holdings America Corporation, Valhalla, NY (US)

(72) Inventors: Yusuke MORI, Madison, WI (US); Tadanori YAMADA, Madison, WI (US); Ryota HABA, Madison, WI (US); Hayato KOBAYASHI, Madison, WI (US); Setsu ENDOH, Madison, WI (US); Kristin WILLIS, Madison, WI (US); Igor GUREVICH, Madison, WI (US)

(73) Assignee: FUJIFILM Holdings America Corporation, Valhalla, NY (US)

(21) Appl. No.: 19/465,957

(22) Filed: Jan. 30, 2026

Related U.S. Application Data
(60) Provisional application No. 63/752,337, filed on Jan. 31, 2025.

(51) Int. Cl. C12N 5/071

(52) U.S. Cl. CPC C12N 5/067; C12N 2500/02; C12N 2500/90; C12N 2501/115; C12N 2501/15; C12N 2501/155; C12N 2501/165; C12N 2501/727; C12N 2506/45

(57) ABSTRACT

Provided herein are methods for producing quiescent hepatic stellate cells (HSCs) from pluripotent stem cells and compositions thereof. In particular aspects, the HSCs are produced without a cell sorting or enrichment step and under feeder-free and serum-free conditions.

### PRIORITY CLAIM

This application claims benefit of priority to U.S. Provisional Application Ser. No. 63/752,337 filed Jan. 31, 2025, the entire contents of which are hereby incorporated by reference.

### BACKGROUND

### 1. Field

The present disclosure relates generally to the field of stem cell biology. More particularly, it concerns the production of hepatic stellate cells, such as from induced pluripotent stem cells (iPSCs).

### 2. Description of Related Art

Hepatic stellate cells (HSC) are liver specific mesenchymal cells. In the body, they exist in two states: quiescent and activated. Under physiological condition, HSC are quiescent and their primary function is the storage of vitamin A and, to a lesser extent, other lipid soluble vitamins. In response to liver injury, HSCs become activated, lose their capacity to store vitamin A, and secrete components of extracellular matrix and growth factors needed for tissue repair. Activation of HSCs is irreversible. Because of the invasive nature of HSC isolation, primary HSCs are overwhelmingly activated, and quiescent HSCs are practically unavailable, preventing the study of mechanisms of activation and assembly of liver organoid models that faithfully recapitulate in vivo liver physiology.

Protocols for generating HSCs from human pluripotent cells exist, however, they either result in the phenotype that is closer to activated HSC than quiescent or require enrichment sorting of the HSC progenitor cells for activated leukocyte cell adhesion molecule (ALCAM, also known as CD166) or the final differentiated HSC population for nerve growth factor receptor (NGFR, also known as CD271) in order to generate quiescent HSCs. Both these enrichment sorting methods considerably decrease the quiescent HSC yields. In addition, methods for generating HSCs comprise the use of feeder cells and/or serum which are not compliant with cell therapy applications. Thus, there is an unmet need for serum-free and/or feeder-free methods of generating quiescent HSCs without enrichment sorting.

### SUMMARY

Provided herein are in vitro methods for producing quiescent hepatic stellate cells (HSCs) comprising (a) culturing pluripotent stem cells (PSCs) in a first media; (b) inducing mesodermal lineage in a second media comprising a bone morphogenic protein (BMP) agonist and a glycogen synthase kinase 3 (GKS3) inhibitor to produce mesodermal cells; (c) culturing the mesodermal cells in the presence of a survival agent to produce aggregates; and (d) differentiating the aggregates in a third media comprising a transforming growth factor beta (TFGβ) inhibitor and a BMP inhibitor to produce a population of quiescent HSCs.

The first media and second media may each, independently, comprise BMP4. The BMP4 may be present at a concentration of 0.5 ng/ml to 5 mg/mL, such as 0.5 to 1 mg/mL, 1 to 2.5 mg/mL, or 2.5 to 5 mg/mL. The second media may further comprise basic fibroblast growth factor (bFGF), such as at a concentration of 1 ng/mL to 10 ng/ml, such as 1 ng/ml to 5 ng/ml or 5 ng/ml to 10 mg/mL.

Steps (a) and (b) may be performed under two-dimensional (2D) conditions. In some aspects, the BMP agonist is SB4, such as at a concentration of 1 μM to 15 μM, such as 1 μM to 5 μM, 5 μM to 10 μM, or 10 μM to 15 μM. The GSK3 inhibitor may be selected from the group consisting of CHIR98014, CHIR99021, BIO, and SB-216763. In particular aspects, the GSK3 inhibitor is CHIR98014, such as at a concentration of 1 μM to 5 μM. In specific aspects, the survival agent is a ROCK inhibitor or myosin II inhibitor, such as blebbistatin. The ROCK inhibitor may be selected from the group consisting of Y27632, chroman, H1152, Fasudil, SR3677, GSK269962, and SB72077B. In specific aspects, the ROCK inhibitor is Y27632. In some aspects, the TGFβ inhibitor is selected from the group consisting of 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinoxaline (SB525334), 2-(5-Benzo[1,3]dioxol-5-yl-2-ieri-butyl-3H-imidazol-4-yl)-6-methylpyridine hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-benzamide hydrate, left-right determination factor (Lefty), 3-(6-Methyl-2-pyridinyl)-N-phenyl-4-(4-quinolinyl)-1H-pyrazole-1-carbothioamide (A 83-01), 4-[4-(2,3-Dihydro-1,4-benzodioxin-6-yl)-5-(2-pyridinyl)-1H-imidazol-2-yl]benzamide (D 4476), 4-[4-[3-(2-Pyridinyl)-1H-pyrazol-4-yl]-2-pyridinyl]-N-(tetrahydro-2H-pyran-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazole-1-ethanol (R 268712) or 2-(3-(6-Methylpyridine-2-yl)-1H-pyrazol-4-yl)-1,5-naphthyridine (RepSox). In particular aspects, the TGFβ inhibitor is SB431542, such as at a concentration of 1 μM to 10 Mn such as 1 μM to 5 μM, 5 μM to 10 M, or 10 μM to 15 μM. In certain aspects, the third media further comprises vascular endothelial growth factor (VEGF), such as at a concentration of 1 ng/mL to 10 ng/ml, such as 1 ng/ml to 5 ng/ml or 5 ng/ml to 10 mg/mL.

In particular aspects, the present method does not comprise enrichment, such as cell sorting, of the quiescent HSCs. For example, enrichment comprises sorting for CD266 or CD271. In some aspects, steps (a) and (b) comprise culturing under hypoxic conditions. In particular aspects, step (c) comprises culturing under hypoxic conditions followed by culturing under normoxic conditions. For example, culturing is under hypoxic conditions for about 3 days (e.g., 1-4 days, 1-2 days, 1-3 days, 1-5 days, 1 day, 2 days, 3 days, or 4 days) followed by culturing under normoxic conditions for about 5 days (e.g., 1-10 days, 2-9 days, 3-8 days, 4-7 days, 4-6 days, 3 days, 4 days, 5 days, 6 days, or 7 days).

In some aspects, the population of quiescent HSCs comprises at least 50% (e.g., at least 55%, 60%, 65%, 70%, 75%, 80%, 85%, 90%, 95%, 96%, 97%, 98%, 99%, or 100%) cells positive for lecithin retinol acyltransferase (LRAT). In certain aspects, the population of quiescent HSCs comprises at least 70% cells positive for lecithin retinol acyltransferase (LRAT). In some aspects, step (c) is performed for less than 10 days, such as for 7 days to 9 days (e.g., 6 days, 7 days, 8 days, or 9 days).

In certain aspects, the method does not comprise mesodermal induction. In particular aspects, the method comprises serum-free and/or feeder-free conditions. The method may comprise xeno-free conditions.

In particular aspects, the expression of LRAT in cells in the population of quiescent HSCs is greater than the expression of ACTA2. In some aspects, the expression of LRATis at least 5-fold greater, such as at least 6-fold greater, at least 7-fold greater, at least 8-fold greater, at least 9-fold greater, or at least 10-fold greater than the expression of ACTA2. In particular aspects, the expression of LRAT is at least 8-fold greater than the expression of ACTA2.

In some aspects, the population of quiescent HSCs express CD271. For example, the population of quiescent HSCs comprises at least 20% (e.g., at least 30%, 40%, 50%, 60%, 70%, 80%, 90%, or 99%) cells positive for CD271. In particular aspects, the population of quiescent HSCs have the capacity to store Vitamin A. In some aspects, the capacity to store Vitamin A is measured by a Vitamin A storage assay.

In additional aspects, the method further comprises cryopreserving the population of quiescent HSCs. In some aspects, thawed quiescent HSCs are cultured as aggregates to maintain quiescence. In particular aspects, the thawed quiescent HSCs retain vitamin A storage capacity and LRAT expression. In additional aspects, the method further comprises activating the population of quiescent HSCs to produce activated HSCs. For example, activating comprises culturing in two-dimensional culture and/or the presence of exogenous TGFβ. In specific aspects, there is a greater accumulation of Vitamin A in activated HSCs as compared to quiescent HSCs. In some aspects, the activated HSCs secrete collagen, such as pro-collagen I alpha. In particular aspects, the activated HSCs secrete collagen at a level at 2-fold higher (e.g., than the population of quiescent HSCs.

Also provided herein are cell populations comprising quiescent hepatic stellate cells (HSCs) from induced pluripotent stem cells, where the cell population is generated under serum-free and/or feeder-free conditions. The cell population may be produced by any of the methods provided herein.

The cell population may not have been sorted for CD266 or CD271. In certain aspects, the cell population has not been enriched. In some aspects, the population of the quiescent HSCs comprises at least 50% (e.g., at least 75%, 80%, 85%, 90%, 95%, 96%, 97%, 98%, 99%, or 100%) cells positive for lecithin retinol acyltransferase (LRAT). In certain aspects, the population of quiescent HSCs comprises at least 70% cells positive for lecithin retinol acyltransferase (LRAT).

In particular aspects, the expression of LRAT in cells in the population of quiescent HSCs is greater than the expression of ACTA2. In some aspects, the expression of LRATis at least 5-fold greater, such as at least 7-fold greater, at least 8-fold greater, at least 9-fold greater, or at least 10-fold greater than the expression of ACTA2. In particular aspects, the expression of LRAT is at least 8-fold greater than the expression of ACTA2.

In some aspects, the composition further comprises hepatocytes and/or Kupfer cells. In certain aspects, co-culture of HSCs with hepatocytes results in an increase in CYP3A4 activity as compared to CYP3A4 activity in HSCs cultured without hepatocytes. In some aspects, co-culture of HSCs with hepatocytes does not result in a decrease in CYP3A4 activity as compared to CYP3A4 activity in HSCs cultured without hepatocytes.

Also provided herein are methods of producing a cell population comprising at least 20% LRAT-protein expressing cells, wherein the cell population has not undergone a cell sorting step. In some aspects, the population of the quiescent HSCs comprises at least 50% (e.g., at least 75%, 80%, 85%, 90%, 95%, 96%, 97%, 98%, 99%, or 100%) cells positive for lecithin retinol acyltransferase (LRAT). In certain aspects, the population of quiescent HSCs comprises at least 70% cells positive for lecithin retinol acyltransferase (LRAT).

In some aspects, the cell population is produced under serum-free conditions. In particular aspects, the cell population is produced under feeder-free conditions. In some aspects, the cell population stores Vitamin A when not exposed to activation stimuli. In particular aspects, the cell population has a higher gene expression of LRAT as compared to ACTA2 when not exposed to activation stimuli.

In certain aspects, the cell population is derived from iPSCs. In some aspects, the cell population comprises at least 20% (e.g., at least 30%, 40%, 50%, 60%, 70%, 80%, or 90%) Brachyury protein expressing cells. In some aspects, the cell population comprises at least 40% (e.g., at least 50%, 60%, 70%, 80%, 90%, or 99%) LRAT positive cells. In some aspects, the cell population secretes collagen when exposed to activation stimuli. In particular aspects, the cell population has a greater expression of ACTA2 as compared to LRAT expression when exposed to activation stimuli.

Also provided herein are cell populations comprising at least 20% LRAT-protein expressing cells, wherein the cell population has not undergone a cell sorting step. In some aspects, the cell population comprises at least 40% (e.g., at least 50%, 60%, 70%, 80%, 90%, or 99%) LRAT-protein expressing cells.

In certain aspects, the cell population is produced under serum-free conditions. In some aspects, the cell population is produced under feeder-free conditions. In particular aspects, the cell population stores Vitamin A when not exposed to activation stimuli. In some aspects, the cell population has a higher gene expression of LRAT as compared to ACTA2 gene expression when not exposed to activation stimuli.

In certain aspects, the cell population is derived from iPSCs. In some aspects, the cell population comprises at least 20% (e.g., at least 30%, 40%, 50%, 60%, 70%, 80%, or 90%) Brachyury protein expressing cells. In some aspects, the cell population comprises at least 40% (e.g., at least 50%, 60%, 70%, 80%, 90%, or 99%) LRAT positive cells. In some aspects, the cell population secretes collagen when exposed to activation stimuli. In particular aspects, the cell population has a greater expression of ACTA2 as compared to LRAT expression when exposed to activation stimuli.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Schematic of HSC protocol. The mesoderm induction medium (Medium 2) comprises an agonist of BMP4 signaling (e.g., SB4) and a GSK3 inhibitor (e.g., CHIR98014). In addition, the mesoderm induction methodology is performed in 3D culture. Key markers routinely used to evaluate differentiation quality by flow cytometry are shown including brachyury after mesoderm induction and CD271 (NGFR), LRAT, and PDGFRβ after HSC induction.

FIG. 2: Quiescence characterization in HSCs generated by the present methods without enrichment sorting in iPSCs (iPSC lines A (left) and B (right) generated from apparently healthy normal (AHN) donors). Flow cytometry plots indicating the purities of markers of quiescent HSCs (CD271, also known as LNGFR or p75NTR, and LRAT (lecithin retinol acyltransferase)) at the time of cryopreservation (Day 11) and 6 days after thaw and 3D (i.e., aggregate) culture are shown. Aggregate culture of HSCs can be used for maintenance of quiescence.

FIGS. 3A-3B: Activation capacity of quiescent HSCs generated by the present methods without enrichment sorting. (FIG. 3A) Maintenance of quiescent HSC marker gene LRAT with concomitant lack of increase in expression of activation associated gene ACTA2 (Actin Alpha 2, Smooth Muscle) during 6 days of aggregate culture and low expression level of LRAT coupled with increase in expression of ACTA2 during 2D culture. (FIG. 3B) Increase in pro-collagen secretion in HSCs cultured in 2D for 6 days compared to HSCs cultured in 3D. 2D culture is a common method of HSC activation in vitro and that pro-collagen secretion is a feature of HSC activation.

FIG. 4: Vitamin A storage capacity in HSCs generated by the present methods without enrichment sorting in iPSCs (iPSC lines A and B). Vitamin A present in cell extracts from HSCs cultured for as aggregates (top) or in 2D (bottom) in media containing no added vitamin A, followed by additional 3 days and 10 μM vitamin A detected by measuring blue fluorescence and normalizing to the total protein level in the extracts. Vitamin A storage capacity is a unique feature of quiescent HSCs and is lost upon activation; retinol (vitamin A) fluoresces blue under ex.360 nm/em.460 nm; 3D culture may be used for maintenance of quiescence in HSCs.

FIG. 5: Schematic of Vitamin A assay for HSCs. Cryopreserved HSC or live (non-cryopreserved HSCs differentiated according to the present methods were plated on ULA 6 well plates at 0.5×10⁶ cells/mL. After 3 days of aggregate (3D) culture in HSC medium 3, Vitamin A was added with daily media exchanges in HSC medium 3. The cell extracts were prepared in RIPA or other mammalian cell extraction buffer. The blue fluorescence was measured, protein assay performed to normalize fluorescence to protein concentration in the extract. Pass criteria is that normalized fluorescence in the cells cultured in the presence of Vitamin A is greater in statistically significant way than in cells cultured in the absence of Vitamin A.

FIGS. 6A-6B: The cell population comprises >20% LRAT-protein expressing cells. (FIG. 6A) Representative flow cytometry scatter plots of the cells analyzed at time of cryopreservation (top) and 6 days post-thaw and culture in the absence of activation stimuli. Note that the number on the scatter plot, e.g., 92.8% on the upper left plot refers to the percentage of cells inside the gate (area outlined). (FIG. 6B) Percentages of LRAT expressing cells as measured by flow cytometry analyzed at the same timepoints and culture conditions as in FIG. 6A. Numbers on top of bars refer to % LRAT-positive cells±standard error of the mean from 3 independent differentiation runs.

FIG. 7: The cell population stores Vitamin A. The cell population stores Vitamin A of which one measure is that the cell population meets the passing criteria of the Vitamin A storage assay. Cells were cultured in the absence of activation stimuli for 3 days to allow recovery from cryopreservation. This was followed by additional 3 days of culture without activation stimuli in the same media excluding (light grey bars) or containing (dark grey bars) vitamin A. Cell extracts were prepared from the whole cell population. Note that culture media does not contain exogenous vitamin A. Both cell lines meet the passing criteria for the assay, that there is a statistically significant difference in the Student's t-test (P<0.05) between the No Vitamin A and 10 μM Vitamin A sets.

FIG. 8: The cell population has higher gene expression of LRAT as compared to ACTA2 when not exposed to activation stimuli. The cell population analyzed were obtained directly from cryopreserved cells (“cryopreservation”) or after 6 days of culture (“Day 6 post-thaw”). RNA was extracted from the cell population and was analyzed for gene expression level of LRAT and ACTA2 by qPCR. Relative gene expression of LRAT (grey bars) and ACTA2 (black bars) genes in total RNA, i.e., extracted from the entire bulk cell population, measured by qPCR from the cells at the time of cryopreservation and after thaw and culture for 6 days in the absence of activation stimuli. Numbers on top of bars refer to relative gene expression. Note that the y-axis is in the logarithmic scale.

FIG. 9: The cell population secretes collagen when exposed to activation stimuli. Pro-collagen I alpha secretion measured by ELISA performed according to manufacturer specifications in media supernatants from cells recovered from cryopreservation and cultured for 6 days in the absence of activation stimuli (3D, solid bars) or activated by 2D culture (2D, checkered bars). Numbers above the bars are measured secretion values normalized to the total protein+/−standard deviations (n=4 per condition) from a representative experiment. ND=not detected, i.e., secretion is below the detection limit of the assay.

FIG. 10: The cell population has higher gene expression of ACTA2 as compared to LRAT when exposed to activation stimuli. Relative gene expression of LRAT (grey bars) and ACTA2 (black bars) genes in total RNA, i.e., extracted from the entire bulk cell population, measured by qPCR from the cells after thaw and culture for 6 days in the absence of activation stimuli (3D) or in the presence of activation stimuli (2D). Numbers on top of bars refer to relative gene expression. Note that the y-axis is in the logarithmic scale.

FIGS. 11A-11B: Cell population comprises more than 20% Brachyury protein expressing cells. (FIG. 11A) Representative flow cytometry scatter plots of the cells analyzed at the end of mesoderm induction in two different iPSC lines. Note that the number on the scatter plot, e.g., 57.0% in the upper left plot, refers to the percentage of cells inside the gate (area outlined). (FIG. 11B) Percentages of Brachyury expressing cells as measured by flow cytometry analyzed at the end of mesoderm induction. Numbers on top of bars refer to % Brachyury-positive cells+ are standard error of the mean from 4 independent differentiation runs.

FIGS. 12A-12B: FACS analysis of the cell population for LRAT and Brachyury. (FIG. 12A) No 1° refers to the cell population that had no primary antibody. This was the negative control. LRAT refers to the use of anti-LRAT antibody to identify cells that express LRAT. (FIG. 12B) No 1° refers to the cell population that had no primary antibody. This was the negative control. Brachyury refers to the use of anti-Brachyury antibody to identify cells that express Brachyury. Note that the number on the scatter plot, e.g., 57.0% on the lower plot of B refers to the percentage of cells inside the gate (area outlined).

FIG. 13: Flow cytometry plots indicating the purity of pan-HSC, i.e., a marker expressed in both quiescent and activated HSCs, marker PDGFRB (Platelet Derived Growth Factor Beta) at the time of cryopreservation (Day 11) and 6 days after thaw and 3D (i.e., aggregate) culture are shown. iPSC line A (left) and iPSC line B (right) are shown.

FIG. 14: CYP3A4 activity in HSCs culture as aggregates either alone or in the presence of HSCs. Equal numbers of hepatocytes were included in both conditions and the aggregates were cultured for 10 days prior to the assay. The CYP3A4 activity in hepatocyte monocultures was set at 1.

### DETAILED DESCRIPTION

Provided herein are methods for producing populations of quiescent HSCs without the need for enrichment sorting as well as the compositions of quiescent HSCs produced by these methods. The quiescence of these cells may be verified by high expression levels of the known marker of quiescent HSCs lecithin retinol acyltransferase (LRAT), the capacity to be activated, and/or the capacity to store vitamin A. The quiescence of the present cells is a property that has not been shown previously in HSCs produced in vitro, such as from iPSCs. Particularly, the present population of HSCs were shown to have a high purity of LRAT, the canonical marker of quiescent HSCs level. This high LRAT expression was observed both at cryopreservation of the HSC as well as 6 days post-thaw when cultured as aggregates.

In particular, the present methods can produce quiescent HSCs without any cell enrichment, such as sorting or purification. This can simplify the overall process, eliminating the need for complex, time-consuming and yield reducing cell sorting procedures. Cell sorting is known to be an inefficient process with a significant fraction of cells having desired marker being lost during the process. By removing this step, the entire workflow becomes more streamlined, efficient, and cost-effective.

Methods are provided herein for the production of quiescent HSCs, such as by differentiation of pluripotent stems cells (e.g., iPSCs) through mesodermal induction under two-dimensional (2D) conditions followed by HSC induction under three-dimensional (3D) or aggregate conditions. The iPSCs may be cultured in a first media (i.e., Media 1) comprising BMP4 and a survival agent. Next, the cells may be cultured in a second media (i.e., Media 2) to continue the mesodermal induction. The media may comprise basic fibroblast growth factor (bFGF), BMP4, Activin A, a BMP agonist, and a Glycogen synthase kinase 3 (GSK3) inhibitor. The mesodermal cells may then be assessed by their expression of brachyury. The cells can then be induced to HSCs by forming aggregates, and culturing in a third media (i.e., Medium 3). The third media may comprise vascular endothelial growth factor (VEGF), a TGFβ inhibitor, a BMP inhibitor, and survival agent, such as ROCK inhibitor or myosin II inhibitor. The HSCs may be characterized by expression of CD271, LRAT, and PDGFRB. For example, the population of HSCs may comprise at least 70% (e.g., at least 75%, 80%, 85%, 90%, 95%, 96%, 97%, 98%, 99%, or 100%) cells positive for LRAT and/or PDGFRB, such as determined by FACS. In addition, the quiescent HSCs can store Vitamin A. This may be assessed by passing a Vitamin A assay, such as shown in FIG. 5.

The present populations of quiescent HSCs may be used to study the mechanisms of HSC activation and in co-culture models with other liver cell types, notably hepatocytes and Kupffer cells (i.e., resident macrophages of the liver), as a more faithful recapitulation of liver physiology than hepatocyte monoculture.

### I. DEFINITIONS

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally occurring cells or artificially modified cells (e.g., fusion cells, genetically modified cells, etc.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs or iPS cells”.

An “embryonic stem (ES) cell” is an undifferentiated pluripotent cell which is obtained from an embryo in an early stage, such as the inner cell mass at the blastocyst stage, or produced by artificial means (e.g. nuclear transfer) and can give rise to any differentiated cell type in an embryo or an adult, including germ cells (e.g. sperm and eggs).

“Induced pluripotent stem cells (iPScs or iPS cells)” are cells generated by reprogramming a somatic cell by expressing or inducing expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

“Programming” is a process that alters the type of progeny a cell can produce. For example, a cell has been programmed when it has been altered so that it can form progeny of at least one new cell type, either in culture or in vivo, as compared to what it would have been able to form under the same conditions without programming. This means that after sufficient proliferation, a measurable proportion of progeny having phenotypic characteristics of the new cell type are observed, if essentially no such progeny could form before programming; alternatively, the proportion having characteristics of the new cell type is measurably more than before programming. This process includes differentiation, dedifferentiation and transdifferentiation.

“Reprogramming” is a process that confers on a cell a measurably increased capacity to form progeny of at least one new cell type, either in culture or in vivo, then it would have under the same conditions without reprogramming. More specifically, reprogramming is a process that confers on a somatic cell a pluripotent potential. This means that after sufficient proliferation, a measurable proportion of progeny having phenotypic characteristics of the new cell type if essentially no such progeny could form before reprogramming; otherwise, the proportion having characteristics of the new cell type is measurably more than before reprogramming.

“Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type. Under certain conditions, the proportion of progeny with characteristics of the new cell type may be at least about 1%, 5%, 25% or more in order of increasing preference.

The term “forward programming” refers to the programming of a multipotent or pluripotent cell, as opposed to a differentiated somatic cell that has no pluripotency, by the provision of one or more specific lineage-determining genes or gene products to the multipotent or pluripotent cell. For example, forward programming may describe the process of programming ESCs or iPSCs to hematopoietic precursor cells or other precursor cells, or to hematopoietic cells or other differentiated somatic cells.

As used herein, the term “subject” or “subject in need thereof” refers to a mammal, preferably a human being, male or female at any age that is in need of a cell or tissue transplantation. Typically, the subject is in need of cell or tissue transplantation (also referred to herein as recipient) due to a disorder or a pathological or undesired condition, state, or syndrome, or a physical, morphological or physiological abnormality which is amenable to treatment via cell or tissue transplantation.

A “survival agent” refers to an agent which promotes and/or supports cell survival when added to cell culture media. For example, Rho-associated kinase (ROCK) inhibitors or Myosin II-specific inhibitors may be used as survival agents. In particular aspects, these survival agents promote aggregation of cells in culture. Exemplary Myosin II-specific inhibitors include but are not limited to Blebbistatin, N-benzyl-p-toluene sulphonamide, and 2,3-Butanedione monoxime.

“Rho-associated kinase inhibitors,” abbreviated as “ROCK inhibitors,” refer to any substance that inhibits or reduces the function of Rho-associated kinase or its signaling pathway in a cell, such as a small molecule, an siRNA, a miRNA, an antisense RNA, or the like. “ROCK signaling pathway,” as used herein, may include any signal processors involved in the ROCK-related signaling pathway, such as the Rho-ROCK-Myosin II signaling pathway, its upstream signaling pathway, or its downstream signaling pathway in a cell. Examples of ROCK inhibitors include, but are not limited to, a Rho-specific inhibitor, a ROCK-specific inhibitor, or a MRLC (myosin regulatory light chain)-specific inhibitor.

As used herein, “administering” shall mean delivering in a manner which is affected or performed using any of the various methods and delivery systems known to those skilled in the art. Administering can be performed, for example, intravenously, orally, via implant, transmucosally, transdermally, intramuscularly, or subcutaneously. Specifically, envisioned is topical administration. “Administering” can also be performed, for example, once, a plurality of times, and/or over one or more extended periods.

“Super donors” are referred to herein as individuals that are homozygous for certain MHC class I and II genes. These homozygous individuals can serve as super donors and their cells, including tissues and other materials comprising their cells, can be transplanted in individuals that are either homozygous or heterozygous for that haplotype. The super donor can be homozygous for the HLA-A, HLA-B, HLA-C, HLA-DR, HLA-DP or HLA-DQ locus/loci alleles, respectively.

An “agonist” or “activator” as used herein refers to drug or chemical that binds to a receptor inside a cell or on its surface and causes activation of the receptor to produce a biological response which is the same or similar to the action as the substance that normally binds to the receptor.

An “agonist of Bone Morphogenetic Protein (BMP4)” signaling as used herein refers to a compound, such as a small molecule, which activates canonical BMP signaling. Exemplary BMP4 agonist include but are not limited to SB4.

A “quiescent hepatic stellate cell (HSC)” as used herein refers to a cell with expression of LRAT, particularly higher expression of LRAT as compared to ACTA2, the capacity to secrete collagen (e.g., pro-collagen I alpha) when activated, and the capacity to store Vitamin A. The capacity to store Vitamin A may be determined by passing a Vitamin A assay, such as shown in FIG. 5. Further, the cell has the capacity to be activated and has higher expression of ACTA2 as compared to LRAT when activated (e.g., an at least 5-fold, 6-fold, 7-fold, or 8-fold or higher expression of LRAT when activated as compared to quiescent conditions). In additional aspects, the quiescent HSC can express CD271, are of mesodermal origin, and are distinct from hepatocytes. The appearance of the quiescent HSCs may be star-shaped.

The “secretion of collagen” or to “secrete collagen” refers to an at least 2-fold (e.g., 3-fold, 4-fold, 5-fold, 6-fold, 7-fold, 8-fold, 9-fold, or 10-fold) increase in the protein level (e.g., protein level as detected by ELISA) when cultured under activation conditions as compared to culture under quiescent conditions. Culturing under “activation conditions” includes but is not limited to culture in 2D and/or in the presence of exogenous TGFβ. Culturing under “quiescent conditions” can refer to culture in 3D.

A “glycogen synthase kinase 3 (GSK3) inhibitor” refers to a compound, such as a small molecule which reduces the activity of the enzyme glycogen synthase kinase-3 (GSK3). Exemplary GSK3 inhibitors include but are not limited to CHIR99021, BIO, and SB-216763.

A “TGFβ inhibitor” refers to a drug, such as a small molecule, that blocks the activity of transforming growth factor-beta (TGF-β), a cytokine that regulates cell growth and differentiation. Exemplary inhibitors include but are not limited to 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinoxaline (SB525334), 2-(5-Benzo[1,3]dioxol-5-yl-2-ieri-butyl-3H-imidazol-4-yl)-6-methylpyridine hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-benzamide hydrate, left-right determination factor (Lefty), 3-(6-Methyl-2-pyridinyl)-N-phenyl-4-(4-quinolinyl)-1H-pyrazole-1-carbothioamide (A 83-01), 4-[4-(2,3-Dihydro-1,4-benzodioxin-6-yl)-5-(2-pyridinyl)-1H-imidazol-2-yl]benzamide (D 4476), 4-[4-[3-(2-Pyridinyl)-1H-pyrazol-4-yl]-2-pyridinyl]-N-(tetrahydro-2H-pyran-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazole-1-ethanol (R 268712) or 2-(3-(6-Methylpyridine-2-yl)-1H-pyrazol-4-yl)-1,5-naphthyridine (RepSox).

“BMP pathway inhibitors” (also referred to herein as BMP inhibitors) may include inhibitors of BMP signaling in general or inhibitors, such as small molecules, specific for BMP1, BMP2, BMP3, BMP4, BMP5, BMP6, BMP7, BMP8a, BMP8b, BMP10 or BMP15. Exemplary BMP inhibitors include 4-(6-(4-(piperazin-1-yl)phenyl) pyrazolo[1,5-a]pyrimidin-3-yl) quinoline hydrochloride (LDN193189), 6-[4-[2-(1-Piperidinyl) ethoxy]phenyl]-3-(4-pyridinyl)-pyrazolo[1,5-a]pyrimidine dihydrochloride (Dorsomorphin), 4-[6-[4-(1-Methylethoxy)phenyl]pyrazolo[1,5-a]pyrimidin-3-yl]-quinoline (DMH1), 4-[6-[4-[2-(4-Morpholinyl) ethoxy]phenyl]pyrazolo[1,5-a]pyrimidin-3-yl]quinoline (DMH-2), and 5-[6-(4-Methoxyphenyl) pyrazolo[1,5-a]pyrimidin-3-yl]quinoline (ML 347).

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

### II. PLURIPOTENT STEM CELLS

Provided herein are methods and compositions for providing quiescent HSCs from pluripotent stem cells. The pluripotent stem cells may be stem cells including but are not limited to, induced pluripotent stem cells and embryonic stem cells.

In particular aspects, the pluripotent stem cells used herein are human embryonic stem cells (ESCs) or induced pluripotent stem cells (iPSCs) which are capable of long-term proliferation in vitro, while retaining the potential to differentiate into all cell types of the body, including the HSCs of the present disclosure. Thus, these cells could potentially provide an unlimited supply of patient-specific functional HSCs for both drug development and therapeutic uses.

### A. Embryonic Stem Cells

The pluripotent stem cells may be embryonic stem cells (ESCs). ES cells are derived from the inner cell mass of blastocysts and have a high in vitro differentiating capability. ES cells can be isolated by removing the outer trophectoderm layer of a developing embryo, then culturing the inner mass cells on a feeder layer of non-growing cells. The replated cells can continue to proliferate and produce new colonies of ES cells which can be removed, dissociated, replated again and allowed to grow. This process of “subculturing” undifferentiated ES cells can be repeated a number of times to produce cell lines containing undifferentiated ES cells (U.S. Pat. Nos. 5,843,780; 6,200,806; 7,029,913). ES cells have the potential to proliferate while maintaining their pluripotency. For example, ES cells are useful in research on cells and on genes which control cell differentiation. The pluripotency of ES cells combined with genetic manipulation and selection can be used for gene analysis studies in vivo via the generation of transgenic, chimeric, and knockout mice.

Methods for producing mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith, 2000). In other methods, mouse ES cells can be grown in serum-free medium in the presence of bone morphogenetic protein and LIF (Ying et al., 2003).

Human ES cells can be produced or derived from a zygote or blastocyst-staged mammalian embryo produced by the fusion of a sperm and egg cell, nuclear transfer, pathogenesis, or the reprogramming of chromatin and subsequent incorporation of the reprogrammed chromatin into a plasma membrane to produce an embryonic cell by previously described methods (Thomson and Marshall, 1998; Reubinoff et al., 2000). In one method, human blastocysts are exposed to anti-human serum, and trophectoderm cells are lysed and removed from the inner cell mass which is cultured on a feeder layer of mouse embryonic fibroblasts. Further, clumps of cells derived from the inner cell mass are chemically or mechanically dissociated, replated, and colonies with undifferentiated morphology are selected by micropipette, dissociated, and replated. In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as MATRIGEL™ or laminin in the presence of “conditioned” medium containing basic fibroblast growth factor (Xu et al., 2001).

ES cells can also be derived from other organisms including rhesus monkey and marmoset by previously described methods (Thomson and Marshall, 1998; Thomson et al., 1995; Thomson and Odorico, 2000; U.S. Pat. No. 5,843,780), as well as from established mouse and human cell lines. For example, established human ES cell lines include MAOI, MA09, ACT-4, HI, H7, H9, H13, H14 and ACT30. As a further example, mouse ES cell lines that have been established include the CGR8 cell line established from the inner cell mass of the mouse strain 129 embryos, and cultures of CGR8 cells can be grown in the presence of LIF without feeder layers.

ES stem cells can be detected by protein markers including transcription factor Oct4, alkaline phosphatase (AP), stage-specific embryonic antigen SSEA-1, stage-specific embryonic antigen SSEA-3, stage-specific embryonic antigen SSEA-4, transcription factor NANOG, tumor rejection antigen 1-60 (TRA-1-60), tumor rejection antigen 1-81 (TRA-1-81), SOX2, or REX1.

### B. Induced Pluripotent Stem Cells

The pluripotent stem cells used herein may be induced pluripotent stem (iPS) cells, commonly abbreviated iPS cells or iPSCs. The induction of pluripotency was originally achieved in 2006 using mouse cells (Yamanaka et al. 2006) and in 2007 using human cells (Yu et al. 2007; Takahashi et al. 2007) by reprogramming of somatic cells via the introduction of transcription factors that are linked to pluripotency. The use of iPSCs circumvents most of the ethical and practical problems associated with large-scale clinical use of ES cells, and patients with iPSC-derived autologous transplants may not require lifelong immunosuppressive treatments to prevent graft rejection.

With the exception of germ cells, any cell can be used as a starting point for iPSCs. For example, cell types could be keratinocytes, fibroblasts, hematopoietic cells, mesenchymal cells, liver cells, or stomach cells. T cells may also be used as a source of somatic cells for reprogramming (U.S. Pat. No. 8,741,648; U.S. Publication No. 2015/0191697). There is no limitation on the degree of cell differentiation or the age of an animal from which cells are collected; even undifferentiated progenitor cells (including somatic stem cells) and finally differentiated mature cells can be used as sources of somatic cells in the methods disclosed herein. iPS cells can be grown under conditions that are known to differentiate human ES cells into specific cell types, and express human ES cell markers including: SSEA-1, SSEA-3, SSEA-4, TRA-1-60, and TRA-1-81.

Somatic cells can be reprogrammed to produce iPS cells using methods known to one of skill in the art. One of skill in the art can readily produce iPS cells, see for example, Published U.S. Patent Application No. 2009/0246875, Published U.S. Patent Application No. 2010/0210014; Published U.S. Patent Application No. 2012/0276636; U.S. Pat. Nos. 8,058,065; 8,129,187; PCT Publication NO. WO 2007/069666 A1, U.S. Pat. Nos. 8,268,620; 8,546,140; 9,175,268; 8,741,648; U.S. Patent Application No. 2011/0104125, and U.S. Pat. No. 8,691,574, which are incorporated herein by reference. Generally, nuclear reprogramming factors are used to produce pluripotent stem cells from a somatic cell. At least three, or at least four, of Klf4, c-Myc, Oct3/4, Sox2, Nanog, and Lin28 may be utilized. Oct3/4, Sox2, c-Myc and Klf4 may be utilized or Oct3/4, Sox2, Nanog, and Lin28.

Mouse and human cDNA sequences of these nuclear reprogramming substances are available with reference to the NCBI accession numbers mentioned in WO 2007/069666 and U.S. Pat. No. 8,183,038, which are incorporated herein by reference. Methods for introducing one or more reprogramming substances, or nucleic acids encoding these reprogramming substances, are known in the art, and disclosed for example, in U.S. Pat. Nos. 8,268,620, 8,691,574, 8,741,648, 8,546,140, in published U.S. Pat. Nos. 8,900,871 and 8,071,369, which are both incorporated herein by reference.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Pub. No. 2003/0211603. In the case of mouse cells, the culture is carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, as would be known to one of skill in the art, may be used with the methods disclosed herein.

Undefined conditions may be used; for example, pluripotent cells may be cultured on fibroblast feeder cells or a medium that has been exposed to fibroblast feeder cells in order to maintain the stem cells in an undifferentiated state. The cell may be cultured in the co-presence of mouse embryonic fibroblasts treated with radiation or an antibiotic to terminate the cell division, as feeder cells. Alternately, pluripotent cells may be cultured and maintained in an essentially undifferentiated state using a defined, feeder-independent culture system, such as a TESR™ medium (Ludwig et al., 2006a; Ludwig et al., 2006b) or E8™/Essential 8™ medium (Chen et al., 2011).

Plasmids have been designed with a number of goals in mind, such as achieving regulated high copy number and avoiding potential causes of plasmid instability in bacteria, and providing means for plasmid selection that are compatible with use in mammalian cells, including human cells. Particular attention has been paid to the dual requirements of plasmids for use in human cells. First, they are suitable for maintenance and fermentation in E. coli, so that large amounts of DNA can be produced and purified. Second, they are safe and suitable for use in human patients and animals. The first requirement calls for high copy number plasmids that can be selected for and stably maintained relatively easily during bacterial fermentation. The second requirement calls for attention to elements such as selectable markers and other coding sequences. Plasmids that encode a marker may be composed of: (1) a high copy number replication origin, (2) a selectable marker, such as, but not limited to, the neo gene for antibiotic selection with kanamycin, (3) transcription termination sequences, including the tyrosinase enhancer and (4) a multicloning site for incorporation of various nucleic acid cassettes; and (5) a nucleic acid sequence encoding a marker operably linked to the tyrosinase promoter. In particular aspects, the plasmids do not comprise a tyrosinase enhancer or promoter. There are numerous plasmid vectors that are known in the art for inducing a nucleic acid encoding a protein. These include, but are not limited to, the vectors disclosed in U.S. Pat. Nos. 6,103,470; 7,598,364; 7,989,425; and 6,416,998, and U.S. application Ser. No. 12/478,154 which are incorporated herein by reference.

An episomal gene delivery system can be a plasmid, an Epstein-Barr virus (EBV)-based episomal vector (U.S. Pat. No. 8,546,140), a yeast-based vector, an adenovirus-based vector, a simian virus 40 (SV40)-based episomal vector, a bovine papilloma virus (BPV)-based vector, or a lentiviral vector. A viral gene delivery system can be an RNA-based or DNA-based viral vector (PCT/JP2009/062911, PCT/JP2011/069588).

### C. Embryonic Stem Cells Derived by Somatic Cell Nuclear Transfer

Pluripotent stem cells for producing the quiescent HSCs may also be prepared by means of somatic cell nuclear transfer, in which a donor nucleus is transferred into a spindle-free oocyte. Stem cells produced by nuclear transfer are genetically identical to the donor nuclei. In one method, donor fibroblast nuclei from skin fibroblasts of a rhesus macaque are introduced into the cytoplasm of spindle-free, mature metaphase II rhesus macaque ooctyes by electrofusion (Byrne et al., 2007). The fused oocytes are activated by exposure to ionomycin, then incubated until the blastocyst stage. The inner cell mass of selected blastocysts are then cultured to produce embryonic stem cell lines. The embryonic stem cell lines show normal ES cell morphology, express various ES cell markers, and differentiate into multiple cell types both in vitro and in vivo.

### D. MHC Haplotype Matching

Major Histocompatibility Complex is the main cause of immune rejection of allogeneic organ transplants. There are three major class I MHC haplotypes (A, B, and C) and three major MHC class II haplotypes (DR, DP, and DQ). The HLA loci are highly polymorphic and are distributed over 4 Mb on chromosome 6. The ability to haplotype the HLA genes within the region is clinically important since this region is associated with autoimmune and infectious diseases and the compatibility of HLA haplotypes between donor and recipient can influence the clinical outcomes of transplantation. HLAs corresponding to MHC class I present peptides from inside the cell and HLAs corresponding to MHC class II present antigens from outside of the cell to T-lymphocytes. Incompatibility of MHC haplotypes between the graft and the host triggers an immune response against the graft and leads to its rejection. Thus, a patient can be treated with an immunosuppressant to prevent rejection. HLA-matched stem cell lines may overcome the risk of immune rejection.

Because of the importance of HLA in transplantation, the HLA loci are usually typed by serology and PCR for identifying favorable donor-recipient pairs. Serological detection of HLA class I and II antigens can be accomplished using a complement mediated lymphocytotoxicity test with purified T or B lymphocytes. This procedure is predominantly used for matching HLA-A and -B loci. Molecular-based tissue typing can often be more accurate than serologic testing. Low resolution molecular methods such as SSOP (sequence specific oligonucleotide probes) methods, in which PCR products are tested against a series of oligonucleotide probes, can be used to identify HLA antigens, and currently these methods are the most common methods used for Class II-HLA typing. High resolution techniques such as SSP (sequence specific primer) methods which utilize allele specific primers for PCR amplification can identify specific MHC alleles.

MHC compatibility between a donor and a recipient increases significantly if the donor cells are HLA homozygous, i.e. contain identical alleles for each antigen-presenting protein. Most individuals are heterozygous for MHC class I and II genes, but certain individuals are homozygous for these genes. These homozygous individuals can serve as super donors and grafts generated from their cells can be transplanted in all individuals that are either homozygous or heterozygous for that haplotype. Furthermore, if homozygous donor cells have a haplotype found in high frequency in a population, these cells may have application in transplantation therapies for a large number of individuals.

Accordingly, iPSCs used in the present methods may be produced from somatic cells of the subject to be treated, or another subject with the same or substantially the same HLA type as that of the patient. In one case, the major HLAs (e.g., the three major loci of HLA-A, HLA-B and HLA-DR) of the donor are identical to the major HLAs of the recipient. In some cases, the somatic cell donor may be a super donor; thus, iPSCs derived from a MHC homozygous super donor may be used to generate HSC. Thus, the HSCs derived from a super donor may be transplanted in subjects that are either homozygous or heterozygous for that haplotype. For example, the HSCs can be homozygous at two HLA alleles such as HLA-A and HLA-B. As such, HSCs produced from super donors can be used in the methods disclosed herein, to produce HSCs that can potentially “match” a large number of potential recipients.

The HLA homozygous HSCs described herein can find use in a broad array of clinical applications involving transplantation of cells and/or tissues. The HLA homozygous HSCs are HLA compatible with a recipient, and therefore can be introduced into the recipient without the need for immunosuppressive therapy, or at least with reduced need for immunosuppressive therapy. A standard immunosuppressive drug regimen costs thousands of dollars per month, and can have undesirable side effects, including infections and cancers that are often life-threatening and expensive to treat. The present HLA homozygous HSCs thus overcome some of the obstacles currently limiting the use of human cells for clinical applications.

### III. DIFFERENTIATION TO HEPATIC STELLATE CELLS

Methods are provided herein for the production of quiescent HSCs, such as by differentiation of pluripotent stems cells (e.g., iPSCs) through mesodermal induction under two-dimensional (2D) conditions followed by HSC induction under three-dimensional (3D) or aggregate conditions. Preferably, the cells do not comprise a reporter gene.

The iPSCs may be cultured in a first media (i.e., Media 1) comprising BMP4 (e.g., 0.5 ng/ml to 5 mg/ml, such as 2 ng/mL) and a survival agent (e.g., a myosin II inhibitor (e.g., Blebbistatin) or a ROCK inhibitor (e.g., Y27632, chroman, H1152, Fasudil, SR3677, GSK269962, or SB72077B) (e.g., 1 μM to 25 μM, such as 10 μM). In addition, the first media may comprise StemPro34™ serum free media or other basal media supplemented with nutrients, such as recombinant human fms-like tyrosine kinase 3 ligand (Flt3L), recombinant human stem cell factor (SCF), recombinant human interleukin 3 (IL-3), recombinant human interleukin 6 (IL-6), and/or recombinant human thrombopoietin (TPO). StemPro34™ media is a serum-free, eukaryotic cell culture medium comprising the ingredients N-acetyl-L-cysteine, 2-mercaptoethanol, and D,L-tocopherol acetate, or derivatives or mixtures thereof (see US20100297090, incorporated herein by reference). The iPSCs may be cultured in this media for about 1-3 days, particularly about 1 day.

Next, the cells may be cultured in a second media (i.e., Media 2) to continue the mesodermal induction. The second media may comprise basic fibroblast growth factor (bFGF) (e.g., 1-10 ng/ml, such as 5 ng/ml), BMP4 (e.g., 10-20 ng/ml, such as 15 ng/ml), Activin A (e.g., 1-5 ng/mL, such as 2.5 ng/ml), a BMP agonist (e.g., SB4, such as 1-15 μM, specifically 10 μM), and a Glycogen synthase kinase 3 (GSK3) inhibitor (e.g., CHIR98014, CHIR99021, BIO, and SB-216763), particularly CHIR98014 (e.g., 1-5 μM, such as about 0.5 μM). The media may comprise the StemPro34™ serum free media or other basal media supplemented with nutrients, such as recombinant human fms-like tyrosine kinase 3 ligand (Flt3L), recombinant human stem cell factor (SCF), recombinant human interleukin 3 (IL-3), recombinant human interleukin 6 (IL-6), and/or recombinant human thrombopoietin (TPO). The media may be changed daily. The culture may be in hypoxic conditions for about 1 to 5 days, such as 1 day, 2 days, 3 days, 4 days or 5 days, particularly about 3 days.

The mesodermal cells may then be assessed by their expression of brachyury. The cells can then be induced to HSCs by forming aggregates, such as at 0.5×10\hat{ }6 cells/mL and culturing in a third media (i.e., Medium 3). The third media may comprise vascular endothelial growth factor (VEGF, such as 1-20 ng/mL, specifically about 10 ng/mL), a TGFβ inhibitor (e.g., SB431542, such as 1-10 μM, specifically about 5.4 μM), a BMP inhibitor (e.g., dorsomorphin, such as 0.1-5 μM, specifically about 0.5 μM), and survival agent, such as ROCK inhibitor (e.g., Y27632, at about 1-25 μM, specifically about 10 μM). The media may be changed every day or every other day. The cells may be induced for about 5-15 days, such as about 6 days, 7 days, 8 days, 9 days, 10 days, 11, days, 12 days, 13 days, 14 days, or 15 days, particularly about 8 days, before harvesting the quiescent HSCs. The culture may initially be under hypoxic conditions, such as about 1-5 days, particularly about 3 days, and then under normoxic conditions, such as about 3-10 days, specifically about 5 days or less than 6 days.

TABLE 1
Exemplary Media Compositions.
StemPro-34 SFM Complete

| Component | Supplier/Cat. # | Amount per L |
|---|---|---|
| StemPro-34 SFM Basal | gibco/10640 | 1 L |
| StemPro-34 SFM | gibco/10641 | 26 mL (2 bottles of |
| Supplement | | supplement) |
| Pen-Strep (100x) | gibco/15140 | 5 mL (0.5X) |

HSC Medium 1

| Component | Supplier/Cat. # | Concentration | |
|---|---|---|---|
| StemPro34 SFM | See Table above | 100% | |
| Complete | | | |
| BMP4 | R&D Systems/314-BP | 2 | ng/mL |
| Y27632 | LKT Labs/Y1000 | 10 | μM |

HSC Medium 2

| Component | Supplier/Cat. # | Concentration | |
|---|---|---|---|
| StemPro34 SFM Complete | See Table above | 100% | |
| Activin A | R&D/338-AC | 2.5 | ng/mL |
| zbFGF (zebrafish fibroblast | Promega/X608X | 5 | ng/mL |
| growth factor) | | | |
| CHIR98014 | SelleckChem/S2745 | 0.5 | μM |
| BMP4 | R&D Systems/314-BP | 15 | ng/mL |
| SB4 | SelleckChem/S0523 | 10 | μM |

HSC Medium 3

| Component | Supplier/Cat. # | Concentration | |
|---|---|---|---|
| StemPro34 SFM | See Table above | 100% | |
| Complete | | | |
| VEGF | Shenandoah/100-44AF | 10 | ng/mL |
| SB431542 | Tocris/1614 | 5.4 | μM |
| Dorsomorphin | Tocris/3093 | 0.5 | μM |
| Y27632 | LKT Labs/Y1000 | 10 | μM |

The HSCs may be characterized by expression of CD271, LRAT, and PDGFRB. For example, the population of HSCs may comprise at least 70% (e.g., at least 75%, 80%, 85%, 90%, 95%, 96%, 97%, 98%, 99%, or 100%) cells positive for LRAT and/or PDGFRB, such as determined by FACS. In particular, the gene expression of LRAT in the cells is greater than the expression gene expression of ACTA2, such as at least 5-fold greater, at least 7-fold greater, at least 8-fold greater, at least 9-fold greater, or at least 10-fold greater, particularly about 8-fold greater.

In addition, the quiescent HSCs can store Vitamin A. This may be assessed by passing a Vitamin A assay, such as shown in FIG. 5.

The quiescent HSCs may be cryopreserved. Once the HSCs are thawed, they may be cultured as aggregates to remain quiescent or cultured as 2D conditions to activate the HSCs.

In particular, the present methods can produce quiescent HSCs without any cell enrichment, such as sorting or purification. This can simplify the overall process, eliminating the need for complex, time-consuming and yield reducing cell sorting procedures. Cell sorting is known to be an inefficient process with a significant fraction of cells having desired marker being lost during the process. By removing this step, the entire workflow becomes more streamlined, efficient, and cost-effective. This can be particularly advantageous in large-scale applications where numerous samples need to be processed rapidly. Furthermore, by bypassing cell sorting, there is a reduced risk of cell damage or loss during the sorting process. Cells may be sensitive to the physical forces encountered during sorting, potentially impacting their viability or altering their characteristics. By avoiding sorting altogether, the integrity and especially functionality of the cells can be better preserved, generating more reliable and accurate data. Another benefit is the potential for higher throughput and increased sample diversity. Without the cell sorting step, a broader range of cell types, with varying characteristics, can be included in the analysis. This enables the examination of heterogeneous cell populations without the constraint of cell sorting criteria. Such inclusive analysis can lead to a more comprehensive understanding of cellular behavior and potentially uncover new insights or correlations that would have otherwise been missed.

Cell sorting necessitates the use of antibodies to recognize target antigens on cell subpopulations. Antibodies for cell sorting are produced by hybridomas, which are a fusion between two cells created in a process that involves fusing mouse B cells with human myeloma cells to create hybrid cells that can produce an unlimited supply of identical antibodies. In these other efforts the fusion cells used to produce the antibodies are often cultured in serum-containing media. Thus, the final HSC-containing cell population is no longer serum-free. The antibody used must be affirmatively declared to be generated in a hybridoma that was grown in serum-free conditions for the final HSC-containing cell population to be labelled serum-free. A further related benefit is that the most commonly used method for cell sorting aimed at generating large numbers of cells-magnetic activated cell sorting-requires the antibodies to be attached to iron containing, e.g., iron-dextran, beads, a fraction of which remains in the sorted population for several days or even over a week, depending on the post-sorting media exchange regimen. The presence of these beads may interfere with downstream assays and, in addition to the presence of antibodies attached to the beads, decreases the utility of the sorted cells for therapeutic applications.

That the cell population has not undergone a cell sorting step can refer to a cell population, starting from the iPSC stage, has not been subjugated to a cell sorting step. There are multiple advantages to avoiding a cell sorting step, such as fluorescence-activated cell sorting (FACS), magnetic-activated cell sorting (MACS), density gradient centrifugation, microfluidic sorting, laser capture microdissection (LCM), and panning.

Lastly, avoiding cell sorting simplifies the experimental workflow, making it more accessible to researchers or laboratories with limited resources, expertise, or access to specialized equipment. This broader accessibility can facilitate the adoption and replication of the disclosed method or technique, fostering collaboration, and accelerating scientific progress. In summary, the benefits of avoiding a cell sorting step in a patent specification include simplifying the process, preserving cell integrity, enabling diverse sample analysis, and increasing accessibility. These advantages not only enhance efficiency and accuracy but also hold the potential to drive scientific advancement in various fields where cell analysis plays an important role.

### A. Cell Culture Conditions

The culturing conditions according to the present disclosure will be appropriately defined depending on the medium and stem cells used. The medium according to the present disclosure can be prepared using a medium to be used for culturing animal cells as its basal medium. As the basal medium, any of E8, TeSR, BME, BGJb, CMRL 1066, Glasgow MEM, Improved MEM Zinc Option, IMDM, Medium 199, Eagle MEM, αMEM, DMEM, Ham, RPMI 1640, and Fischer's media, as well as any combinations thereof can be used, but the medium is not particularly limited thereto as far as it can be used for culturing animal cells.

In particular aspects, the medium according to the present disclosure is a serum-free medium. The serum-free medium refers to media with no unprocessed or unpurified serum and accordingly, can include media with purified blood-derived components or animal tissue-derived components (such as growth factors). The medium according to the present disclosure may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example. Alternatively, any commercially available materials can be used for more convenience. The commercially available materials include knockout Serum Replacement (KSR), Chemically-defined Lipid concentrated (Gibco), and Glutamax (Gibco).

The medium of the present disclosure can also contain fatty acids or lipids, amino acids (such as non-essential amino acids), vitamin(s), growth factors, cytokines, antioxidant substances, 2-mercaptoethanol, pyruvic acid, buffering agents, and inorganic salts. The concentration of 2-mercaptoethanol can be, for example, about 0.05 to 1.0 mM, and particularly about 0.1 to 0.5 mM, but the concentration is particularly not limited thereto as long as it is appropriate for culturing the stem cell(s).

A culture vessel used for culturing the stem cell(s) can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, roller bottle, and bioreactors, such as PBS500 and/or PBS3, as long as it is capable of culturing the stem cells therein. The stem cells may be culture in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, 2000 ml, or any range derivable therein, depending on the needs of the culture. The culture vessel may be a bioreactor, which may refer to any device or system that supports a biologically active environment. The bioreactors may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, and fibronectin and mixtures thereof for example MATRIGEL™, and lysed cell membrane preparations (Klimanskaya et al., 2005).

Other culturing conditions can be appropriately defined. For example, the culturing temperature can be about 30 to 40° C., for example, at least or about 31, 32, 33, 34, 35, 36, 37, 38, 39° C. but particularly not limited to them. The CO₂ concentration can be about 1 to 10%, for example, about 2 to 5%, or any range derivable therein. The oxygen tension can be at least or about 1, 5, 8, 10, 20%, or any range derivable therein.

The methods of the present disclosure can be also used for a suspension culture of stem cells, including suspension culture on carriers (Fernandes et al., 2007) or gel/biopolymer encapsulation (US20070116680). The term suspension culture of the stem cells means that the stem cells are cultured under non-adherent condition with respect to the culture vessel or feeder cells (if used) in a medium. The suspension culture of stem cells includes a dissociation culture of stem cells and an aggregate suspension culture of stem cells. The term dissociation culture of stem cells means that suspended stem cells is cultured, and the dissociation culture of stem cells include those of single stem cell or those of small cell aggregates composed of a plurality of stem cells (for example, about 2 to 400 cells). When the aforementioned dissociation culture is continued, the cultured, dissociated cells form a larger aggregate of stem cells, and thereafter an aggregate suspension culture can be performed. The aggregate suspension culture includes an embryoid culture method (see Keller et al., 1995), and a SFEB method (Watanabe et al., 2005); International Publication No. 2005/123902). The methods of the present disclosure can significantly improve the survival rate and/or differentiation efficiency of stem cells in a suspension culture.

Bioreactors can be grouped according to general categories including: static bioreactors, stirred flask bioreactors, rotating wall vessel bioreactors, hollow fiber bioreactors and direct perfusion bioreactors. Within the bioreactors, cells can be free, or immobilized, seeded on porous 3-dimensional scaffolds (hydrogel). In certain aspects, the bioreactor is a suspension bioreactor for efficient mixing with homogeneous particle suspension and low shear stress.

The methods disclosed utilized herein may use all GMP compatible materials and be scaled to multiple (e.g., 3L) bioreactor manufacturing batches to yield the purity and cell numbers needed for dendritic cell therapy development.

Serum-free media offer numerous advantages in cell culture, making them valuable for various applications, including therapeutic cell production. These advantages not only enhance the safety and regulatory compliance but also contribute to the reproducibility, scalability, and cost-effectiveness of the cell culture process. Serum-free media can eliminate the risk of introducing contaminants, adventitious agents, or pathogens that may be present in serum. This ensures the safety and purity of cell culture processes, meeting the stringent regulatory requirements for therapeutic cell production. The use of serum-free media aligns with regulatory guidelines for the manufacture of biologics and cell-based therapies. Regulatory agencies recommend or require serum-free or xeno-free culture conditions to minimize the risks associated with serum, ensuring compliance and facilitating the regulatory approval of cell therapy products. Serum-free media can provide a defined and controlled environment, minimizing variability associated with different serum batches, origins, and concentrations. This leads to more consistent and reproducible cell culture conditions, important for obtaining reliable and comparable results in research and large-scale production. Serum-free media can be tailor-made, supplemented with defined growth factors, or specific nutrients that support optimal cell growth and viability. This leads to improved cell expansion, proliferation, and maintenance of desired cell phenotypes, for successful therapeutic cell manufacturing. It can also enable efficient scale-up of cell culture processes in bioreactors or production systems. They provide consistent and reliable cell yields required for large-scale commercial applications, addressing the need for industrialization and commercial viability of therapeutic cell production. Finally, it can potentially reduce manufacturing costs by eliminating the need for expensive serum components. Moreover, the absence of serum simplifies the cell culture process by eliminating steps such as serum heat inactivation or lipid depletion, making it a more cost-effective and straightforward approach.

For a cell population to be serum-free and/or feeder-free, every component at every step in the entire process of manufacture is serum-free and/or feeder-free. Any component that is not asserted to be serum-free and/or feeder-free cannot be assumed to be serum-free and/or feeder-free, and would not be acceptable to be labelled as such by the regulatory agencies. Additionally, OP9 cells are derived using serum-containing media. Thus, if OP9 cells were used as feeder cells at any step upstream in manufacture, any downstream cell population would not be serum-free.

Xeno-free media provide several advantages in cell culture, particularly for applications involving the production of therapeutic cells These advantages include the enhanced safety, regulatory compliance, consistency, reproducibility, and clinical relevance provided by xeno-free media. Xeno-free media can eliminate the risk of introducing potentially harmful contaminants or adventitious agents derived from animal-derived serum products. This ensures the safety and purity of cell culture processes, meeting the stringent regulatory requirements for therapeutic cell production and minimizing potential adverse effects on patients. The use of xeno-free media aligns with regulatory guidelines for the manufacturing of biologics and cell-based therapies. Regulatory agencies often recommend or require xeno-free or serum-free culture conditions to reduce the risks associated with animal-derived components. By using xeno-free media, cell therapy products can meet regulatory standards, facilitating their approval and commercialization. It can provide a defined and controlled environment, reducing variability associated with lot-to-lot variations in animal-derived serum. This leads to more consistent and reproducible cell culture conditions, important for obtaining reliable and comparable results in research and large-scale production of therapeutic cells. Th xeno-free media allow for better standardization and comparability of cell culture processes. By eliminating the variability introduced by animal-derived serum, xeno-free media can provide a more controlled and consistent environment, leading to robust and standardized manufacturing protocols. This enhances process scalability, reproducibility, and clinical translation of cell therapy products. Xeno-free media can significantly reduce the risk of introducing xenogeneic components that might trigger an immune response or generate adverse reactions in patients receiving cell therapies. By excluding animal-derived materials, xeno-free media minimize immunogenicity concerns and enhance the safety profile of therapeutic cell products. It can facilitate the efficient scale-up of cell culture processes in bioreactors or production systems. They provide consistent and reliable cell growth and expansion, addressing the need for large-scale commercial manufacturing of therapeutic cells. The scalability offered by xeno-free media contributes to the industrialization and wider availability of cell-based therapies. Finally, xeno-free media streamline cell culture processes by eliminating the need for animal-derived serum and associated steps such as serum heat inactivation or lipid depletion. This simplifies and reduces the complexity of the culture protocols, providing a more straightforward and cost-effective approach to therapeutic cell production.

Feeder-free culture can eliminate the need for animal-derived feeder cells, leading to increased experimental reproducibility and standardization. This system allows for higher cell growth rates and reduces the risk of contamination. Feeder cells may contaminate the stem cell population with non-stem cell derivatives, posing challenges for downstream applications. Feeder-free culture enables the isolation of homogenous stem cell populations, reducing the risk of unwanted cellular heterogeneity. Feeder-free culture enables large-scale expansion of stem cells, facilitating the production of ample quantities required for various applications, such as cell therapies and drug discovery. This scalability is important for meeting the growing demand for stem cell-based products. Animal-derived feeder cells may introduce unknown factors that can interfere with the genetic stability of cultured stem cells. Feeder-free culture eliminates this risk, ensuring the safety and integrity of the stem cell population, thus making it suitable for clinical applications. The absence of feeders and undefined components in feeder-free culture allows for greater control over the stem cell environment. This leads to reduced batch-to-batch variability, improving experimental reproducibility and the validity of research findings. Feeder-free culture systems can utilize defined or serum-free media, which enables better control over cell culture conditions. This compatibility with defined media facilitates the standardization of protocols, leading to more consistent research outcomes and easier transition to clinical applications. Feeder-free culture can eliminate the dependence on feeder cell preparation and maintenance, saving time and labor. This simplified workflow increases efficiency and optimizes resource utilization, making it a cost-effective approach for stem cell research and production. The use of feeder-free culture systems reduces the reliance on animal-derived materials, addressing ethical concerns associated with the use of animal products in research. This promotes the development and commercialization of stem cell products with a broader societal acceptance. Feeder-free culture systems contribute to the standardization of stem cell research protocols, enabling better comparison and reproducibility between different laboratories. This advancement helps establish consensus and best practices in the field, fostering collaborations and driving progress in stem cell research.

One of the major challenges in transitioning from serum-based cell culture to serum-free media is maintaining cell viability and proliferation rates. Serum contains a wide array of nutrients, growth factors, hormones, and proteins that play vital roles in supporting cell growth and maintaining cell health. Removing serum from the culture media can lead to a reduction in the availability of these essential components, potentially resulting in decreased cell viability, compromised cell proliferation, and alteration of the cell phenotype.

Without the presence of serum, cells may not receive certain nutrients or growth factors required for their survival. The absence of essential trace elements and micronutrients present in serum can further impact cell growth and functionality. Thus, ensuring adequate provision of these key components in serum-free media is critical to address the challenge of maintaining robust cell viability and proliferation rates. Moreover, provision of these key factors is an important consideration in maintaining the appropriate phenotype of the cells being cultured.

Another difficulty in adapting cell culture to serum-free media is achieving consistent and reproducible results across different laboratories. Serum is a complex and poorly defined mixture, with its composition varying between batches and suppliers. This inherent variability poses a challenge in reproducing experimental results when serum is used as a supplement in cell culture.

By transitioning to serum-free media, researchers aim to eliminate the batch-to-batch variability associated with serum. However, adapting cells to serum-free conditions often results in significant inter-laboratory variability due to the inability to experimentally reproduce the exact composition of serum. Consequently, it becomes important to formulate defined media compositions that can offer consistent and reproducible growth environments for various cell types.

Serum contains numerous signaling molecules that play important roles in regulating cellular behavior, differentiation, gene expression, and maintaining specific cell phenotypes. These complex signaling pathways are intricately linked to the presence of various growth factors and cytokines present in serum. Transitioning to serum-free media can disrupt these signaling networks, potentially impacting cell functionality and altering cellular responses.

To address this challenge, it is necessary to identify and incorporate appropriate substitutes or supplements in serum-free media that can mimic the signaling molecules present in serum. Determining the specific factors required for each cell type is important to retain the desired cellular behavior and avoid unintended consequences when transitioning to serum-free conditions.

Developing an effective and optimized serum-free media formulation that supports the growth and functionality of specific cell lines is a complex task. Each cell type may have different nutritional requirements, growth factor dependencies, and specific sensitivities to culture conditions. The absence of serum necessitates the identification and optimization of media components to meet the specific needs of the cells being cultured.

Understanding the nutritional requirements of the desired cell lines and systematically optimizing the media composition is a time-consuming process. Formulating defined media that can replicate the functionalities provided by serum while avoiding the limitations associated with serum supplementation is important to achieving successful adaptation in serum-free conditions.

In conclusion, the difficulties in adapting cell culture from serum to serum-free media lie in maintaining cell viability and proliferation rates, achieving reproducibility and consistency, overcoming disruptions to complex signaling pathways, and optimizing a well-defined formulation tailored to different cell types. The present methods can address these challenges by facilitating the adoption of serum-free media, improving cell culture outcomes in various scientific research, biotechnological, and therapeutic applications.

Activation stimuli include but are not limited to exposure of the cells to pro-fibrotic growth factors. These include transforming growth factor-beta (TGF-β), platelet-derived growth factor (PDGF), fibroblast growth factors (FGFs), and angiotensin II. Further stimuli are exposure of the cells to Inflammatory cytokines These include interleukin-1 beta (IL-1β) and tumor necrosis factor-alpha (TNF-α); exposure of the cells to Lipopolysaccharides (LPS); and exposure of the cells to mechanical stress. Examples of mechanical stress that can be applied to activate LRAT+ cells include: Cyclic stretching: Applying rhythmic mechanical stretching to the cells using specialized equipment such as a cell stretcher or a bioreactor can mimic the increased intrahepatic pressure observed in liver fibrosis; Compression: Subjecting the cells to pressure or compression using a compression device or through culturing them on micropatterned substrates can activate hepatic stellate cells; Shear stress: Exposing the cells to fluid flow or laminar shear stress using microfluidic devices or parallel plate flow chambers can induce activation of hepatic stellate cells; and Substrate stiffness: Culturing the cells on substrates with varying stiffness levels, ranging from soft to stiff, can simulate the mechanical rigidity changes in liver tissue during fibrosis and activate hepatic stellate cells. This includes culturing the cells on a 2-dimensional (2D) surface.

### B. Characterization of HSCs

The cells obtained according to the present methods can be characterized according to a number of phenotypic criteria.

Tissue-specific markers can be detected using any suitable immunological technique—such as flow immunocytometry or affinity adsorption for cell-surface markers, immunocytochemistry (for example, of fixed cells or tissue sections) for intracellular or cell-surface markers, Western blot analysis of cellular extracts, and enzyme-linked immunoassay, for cellular extracts or products secreted into the medium. Expression of an antigen by a cell is said to be antibody-detectable if a significantly detectable amount of antibody will bind to the antigen in a standard immunocytochemistry or flow cytometry assay, optionally after fixation of the cells, and optionally using a labeled secondary antibody.

Quiescent HSCs can be characterized by the expression of LRAT, CD271, and PDGFRB (platelet-derived growth factor receptor beta). Further assays can be performed to determine the capacity to be activated and the capacity to store Vitamin A. LRAT expression may be determined by flow cytometry using a mouse monoclonal antibody directed against human LRAT and gated. In addition, αSMA (by expression of ACTA2) and GFAP are both associated with activated HSCs and should have low or essentially no expression in quiescent HSCs. COLIA1 gene expression is also associated with activated HSCs and should have low to essentially no expression in quiescent cells and increase as a result of exposure to activation stimuli. Other markers that may be assessed include desmin, PDGFRB, ALCAM, and PCDH7.

A cell population that comprises at least or more than 20% LRAT-protein expressing cells means that at least 20% of the cells in the cell population express LRAT. One way to measure this is by FACS. Other methods contemplated include in situ hybridization, immunohistochemistry, immunofluorescence, and western blot analysis. Analyzing the cell population via FACS shows that at least 20% of the cells are identified as LRAT-positive. A cell population that comprises at least 40% LRAT-protein expressing cells means that at least 40% of the cells in the cell population express LRAT. A cell population that comprises at least 60% LRAT-protein expressing cells means that at least 60% of the cells in the cell population express LRAT. A cell population that comprises at least 80% LRAT-protein expressing cells means that at least 80% of the cells in the cell population express LRAT. Another way is to detect activity of the LRAT promoter, for example by analyzing LRAT mRNA levels. This can be performed via qPCR or single-cell RNA hybridization or sequencing. Another way is to use a reporter gene or tag under the control of the LRAT promoter or gene enhancers. A cell population that comprises at least 20% LRAT-protein expressing cells storing Vitamin A means that the cells in this cell population sequester a measurable amount of Vitamin A from culture media. One method to detect this Vitamin A-storing behavior is by using the Vitamin A storage assay. A cell population is said to store Vitamin A when it meets the passing criteria for the Vitamin A storage assay.

A cell population that has a higher expression of LRAT as compared to ACTA2 refers to the relative gene expression of LRAT in a cell population not exposed to an activation stimulus as compared a cell population exposed to an activation stimulus is greater than the relative gene expression of ACTA2 in a cell population not exposed to an activation stimulus as compared to a cell population exposed to an activation stimulus. A cell population that has a higher expression of ACTA2 as compared to the expression of LRAT refers to the relative ACTA2 gene expression in a cell population exposed to activation stimuli (i.e., activated cells) compared to a cell population not exposed to activation stimuli (i.e., quiescent cells) is greater than the relative LRAT gene expression in the in a cell population exposed to activation stimuli (i.e., activated cells) compared to a cell population not exposed to activation. A cell population the secretes pro-collagen I alpha when exposed to activation stimuli refers to a cell population that secretes a greater level of the pro-collagen I alpha protein when exposed to activation stimuli than when not exposed to activation stimuli. This can be measured by ELISA (e.g., Human Pro-collagen I alpha ELISA). A cell population that comprises at least 20% Brachyury protein expressing cells refers to at least 20% of the cells in the cell population being stained with the antibody directed against human Brachyury protein. A cell population that comprise at least 40% Brachyury protein expressing cells refers to at least 40% of the cells in the cell population being stained with the antibody directed against human Brachyury protein. This can be detected by fluorescence activated cell sorting (FACS) performed as described with the Brachyury antibody either directly conjugated to a fluorochrome or labeled with a secondary antibody conjugated to a fluorochrome and gated based on the negative control cell population. The negative control cell population is not stained with the Brachyury antibody.

The expression of tissue-specific gene products can also be detected at the mRNA level by Northern blot analysis, dot-blot hybridization analysis, or by reverse transcriptase-initiated polymerase chain reaction (RT-PCR) using sequence-specific primers in standard amplification methods using publicly available sequence data (GenBank). Expression of tissue-specific markers as detected at the protein or mRNA level is considered positive if the level is at least or about 2-, 3, 4-, 5-, 6-, 7-, 8-, or 9-fold, and more particularly more than 10-, 20-, 30, 40-, or 50-fold above that of a control cell, such as an undifferentiated pluripotent stem cell or other unrelated cell type.

Once markers have been identified on the surface of cells of the desired phenotype, they can be used for immunoselection to further enrich the population by techniques such as immunopanning or antibody-mediated fluorescence-activated cell sorting.

Where derived from an established line of pluripotent stem cells, the cell populations and isolated cells of the present disclosure can be characterized as having the same genome as the line from which they are derived. This means that the chromosomal DNA will be over 90% identical between the pluripotent stem cells and the HSCs, which can be inferred if the HSCs are obtained from the undifferentiated line through the course of normal mitotic division. The characteristic that HSCs are derived from the parent cell population is important in several respects. In particular, the undifferentiated cell population can be used for producing additional cells with a shared genome—either a further batch HSCs, or another cell type that may be useful in therapy.

### IV. METHODS OF USE

The HSCs provided by methods and compositions of certain aspects can be used in a variety of applications. These include but are not limited to transplantation or implantation of the cells in vivo; screening cytotoxic compounds, carcinogens, mutagens growth/regulatory factors, pharmaceutical compounds, etc., in vitro; elucidating the mechanism of diseases and injuries; studying the mechanism by which drugs and/or growth factors operate; diagnosing and monitoring cancer in a patient; gene therapy; and the production of biologically active products.

HSCs of the present disclosure can be used commercially to screen for factors (such as solvents, small molecule drugs, peptides, oligonucleotides) or environmental conditions (such as culture conditions or manipulation) that affect the characteristics of such cells and their various progeny. They may also be used to treat or prevent liver disease.

Cytotoxicity can be determined in the first instance by the effect on cell viability, survival, morphology, and the expression of certain markers and receptors. Effects of a drug on chromosomal DNA can be determined by measuring DNA synthesis or repair. [3H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread. The reader is referred to Vickers (pp 375-410 in In vitro Methods in Pharmaceutical Research, Academic Press, 1997) for further elaboration.

A method of treatment may include treatment of a cancer in a subject. Examples of cancers can include acute lymphoblastic leukemia, acute myeloid leukemia, adrenocortical carcinoma, basal-cell carcinoma, bile duct cancer, bladder cancer, bone tumor, osteosarcoma/malignant fibrous histiocytoma, brainstem glioma, brain tumor, cerebellar astrocytoma, cerebral astrocytoma/malignant glioma, ependymoma, medulloblastoma, supratentorial primitive neuroectodermal tumors, breast cancer, bronchial adenomas/carcinoids, Burkitt's lymphoma, carcinoid tumor, cervical cancer, chronic lymphocytic leukemia, chronic myelogenous leukemia, chronic myeloproliferative disorders, colon cancer, cutaneous T-cell lymphoma, endometrial cancer, ependymoma, esophageal cancer, Ewing's sarcoma, intraocular melanoma, retinoblastoma, gallbladder cancer, gastric cancer, gastrointestinal carcinoid tumor, gastrointestinal stromal tumor, germ cell tumor, glioma, childhood visual pathway and hypothalamic, Hodgkin lymphoma, melanoma, islet cell carcinoma, Kaposi sarcoma, renal cell cancer, laryngeal cancer, leukemia, lymphomas, mesothelioma, neuroblastoma, non-Hodgkin lymphoma, oropharyngeal cancer, osteosarcoma, ovarian cancer, pancreatic cancer, parathyroid cancer, pharyngeal cancer, pituitary adenoma, plasma cell neoplasia, prostate cancer, renal cell carcinoma, retinoblastoma, sarcoma, testicular cancer, thyroid cancer, and uterine cancer.

### A. Pharmaceutical Compositions

The present disclosure further provides compositions comprising populations of HSCs. The compositions may comprise pharmaceutically acceptable carriers and diluents. The compositions may further comprise components that facilitate engraftment. Compositions comprising these populations are useful for cell and tissue replacement and repair, and for generating populations of HSCs in vitro and in vivo. The compositions may be formulated as a medicament or delivery device for treating an immune condition.

The HSCs of the present disclosure can be supplied in the form of a pharmaceutical composition, comprising an isotonic excipient prepared under sufficiently sterile conditions for human administration. In certain aspects, it may be desirable to disperse the cells using a protease or by gentle mechanical manipulation into a suspension of single cells or smaller clusters. To reduce the risk of cell death upon engraftment, the cells may be treated by heat shock or cultured with about 0.5 U/mL erythropoietin about 24 hours before administration.

For general principles in medicinal formulation, the reader is referred to Cell Therapy: Stem Cell Transplantation, Gene Therapy, and Cellular Immunotherapy, 1996; and Hematopoetic Stem Cell Therapy, 2000. Choice of the cellular excipient and any accompanying elements of the composition will be adapted in accordance with the route and device used for administration.

This disclosure also includes a reagent system, comprising a set or combination of cells that exist at any time during manufacture, distribution, or use. The cell sets comprise any combination of two or more cell populations described in this disclosure, exemplified but not limited to a type of differentiated cell, in combination with undifferentiated pluripotent stem cells or other differentiated cell types, often sharing the same genome. Each cell type in the set may be packaged together, or in separate containers in the same facility, or at different locations, at the same or different times, under control of the same entity or different entities sharing a business relationship.

Pharmaceutical compositions of this disclosure may optionally be packaged in a suitable container with written instructions for a desired purpose.

### B. Therapeutic Uses

The cells provided in certain aspects of this present disclosure can be used for therapy of any subject in need thereof.

To determine the suitability of cell compositions for therapeutic administration, the cells can first be tested in a suitable animal model. At one level, cells are assessed for their ability to survive and maintain their phenotype in vivo. Cell compositions are administered to immunodeficient animals (such as NUDE rats, or animals rendered immunodeficient chemically or by irradiation). Tissues are harvested after a period of engraftment, and assessed as to whether pluripotent stem cell-derived cells are still present.

Other methods to track cells in vivo may be by administering cells that express a detectable label (such as green fluorescent protein, or β-galactosidase); that have been prelabeled (for example, with BrdU or [³H]thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

After adequate testing, differentiated cells of this disclosure can be used for tissue reconstitution or regeneration in a human patient or other subject in need of such treatment. The cells are administered in a manner that permits them to graft or migrate to the intended tissue site and reconstitute or regenerate the functionally deficient area.

Where desirable, the patient receiving an allograft of pluripotent stem cell-derived HSCs can be treated to reduce immune rejection of the transplanted cells. Methods contemplated include the administration of traditional immunosuppressive drugs like cyclosporin A (Dunn et al., Drugs 61:1957, 2001), or inducing immunotolerance using a matched population of pluripotent stem cell-derived cells (WO 02/44343; U.S. Pat. No. 6,280,718; WO 03/050251). Another approach is to adapt the HSCs to decrease the amount of uric acid produced by the cells upon transplantation into a subject, for example, by treating them with allopurinol. Alternatively or in conjunction, the patient is prepared by administering allopurinol, or an enzyme that metabolizes uric acid, such as urate oxidase (PCT/US04/42917).

With respect to the therapeutic methods of the present disclosure, it is not intended that the administration of HSCs to a mammal be limited to a particular mode of administration, dosage, or frequency of dosing; the present disclosure contemplates all modes of administration, including intramuscular, intravenous, intrarticular, intralesional, subcutaneous, or any other route sufficient to provide a dose adequate to prevent or treat a disease. The HSCs may be administered to the mammal in a single dose or multiple doses. When multiple doses are administered, the doses may be separated from one another by, for example, one week, one month, one year, or ten years. One or more growth factors, hormones, interleukins, cytokines, small molecules or other cells may also be administered before, during, or after administration of the cells to further bias them towards a particular cell type.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Production of HSCs from iPSCs

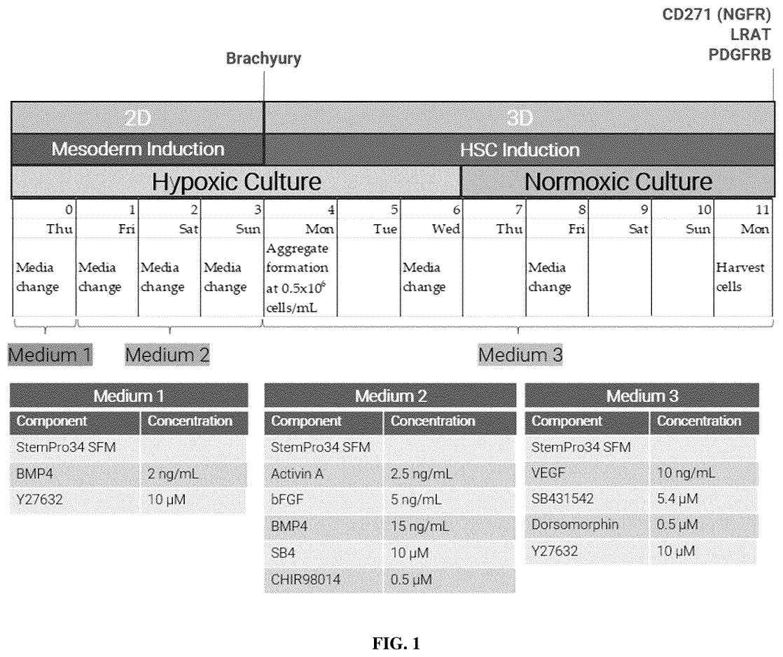

Quiescent HSCs were produced by mesodermal induction (FIG. 1). iPSCs cultured in Media 1 comprising BMP4 (2 ng/mL), a survival agent, such as a ROCK inhibitor (e.g., Y27632 (10 μM)) or a myosin II inhibitor (e.g., Blebbistatin), and STEMPRO34™ serum free media. The iPSCs were cultured for about 1 day.

Next, the cells were cultured in Media 2 to continue the mesodermal induction. The media comprised bFGF (5 ng/ml), BMP4 (15 ng/ml), a BMP agonist (e.g., SB4, 10 μM), and a Glycogen synthase kinase 3 (GSK3) inhibitor (e.g., CHIR99021, 0.5 μM), and STEMPRO34™ serum free media. The media was changed daily. The culture was under hypoxic conditions for about 3 days. The mesodermal cells were then assessed by their expression of brachyury.

The cells were then be induced to HSCs by forming aggregates, such as at 0.5×10\hat{ }6 cells/mL and culturing in Medium 3. The media comprised VEGF (10 ng/mL), a TGFβ inhibitor (e.g., SB431542, 5.4 μM), a BMP inhibitor (e.g., dorsomorphin, 0.5 μM), and survival agent, such as ROCK inhibitor (e.g., Y27632, 10 μM) or myosin II inhibitor. The media was changed every other day for about 8 days, before harvesting the quiescent HSCs. The culture may initially be under hypoxic conditions, such as about 3 days, and then under normoxic conditions, such as about 5 days.

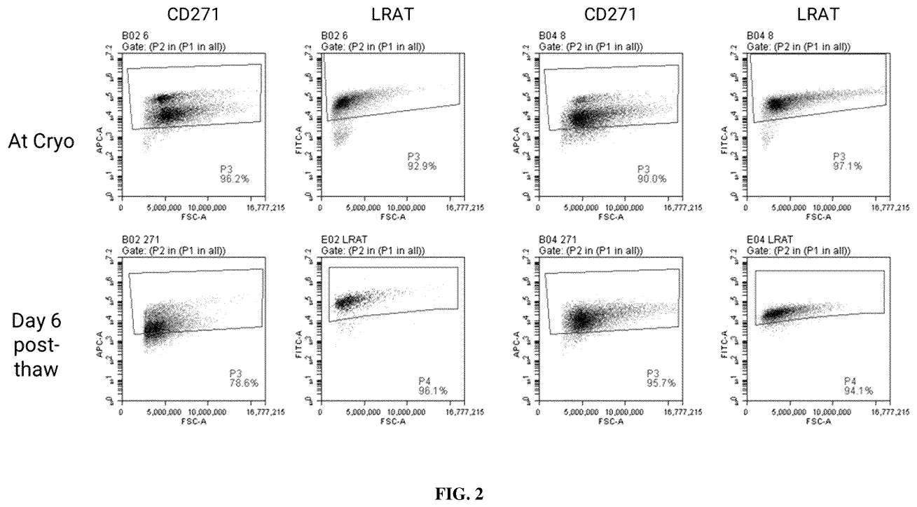

The HSCs were characterized for their quiescence (FIG. 2). Flow cytometry plots indicating the purities of markers of quiescent HSCs (CD271, also known as LNGFR or p75NTR, and LRAT (lecithin retinol acyltransferase)) at the time of cryopreservation (Day 11) and 6 days after thaw and 3D (i.e., aggregate) culture in the absence of activation stimuli.

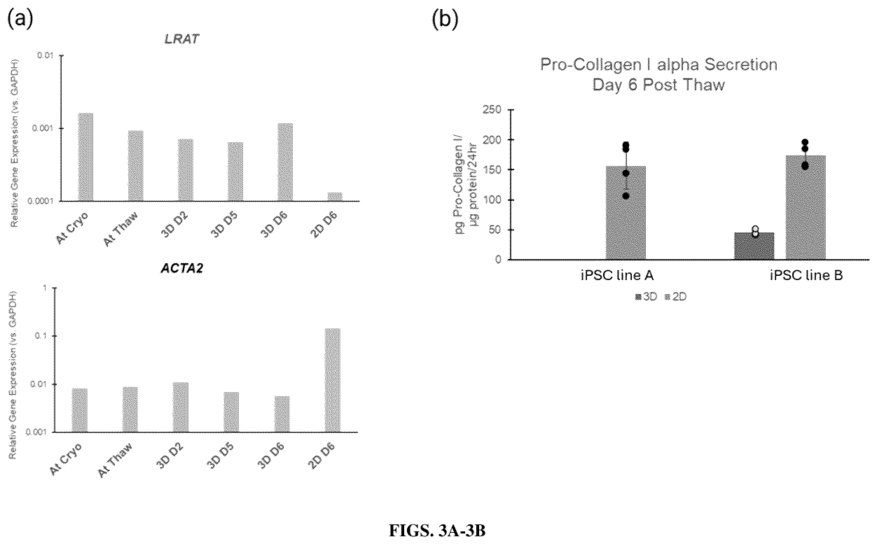

The activation capacity of the quiescent HSCs generated was assessed (FIG. 3). Maintenance of the quiescent HSC marker gene LRAT with a concomitant lack of increase in expression of activation associated gene ACTA2 (Actin Alpha 2, Smooth Muscle) during 6 days of aggregate culture was observed along with a low expression level of LRAT coupled with an increase in expression of ACTA2 during the 2D culture. An increase in pro-collagen secretion in HSCs cultured in 2D for 6 days compared to HSCs cultured in 3D was also observed.

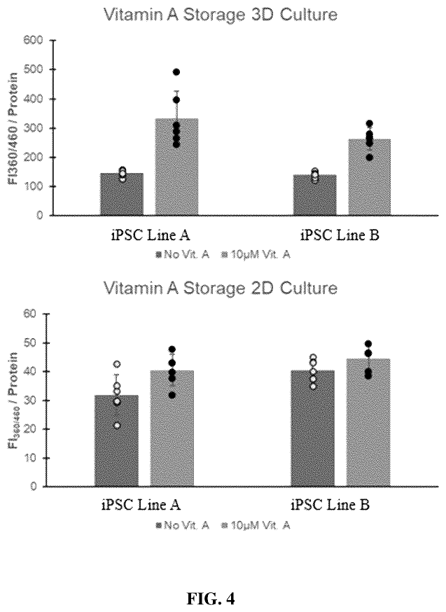

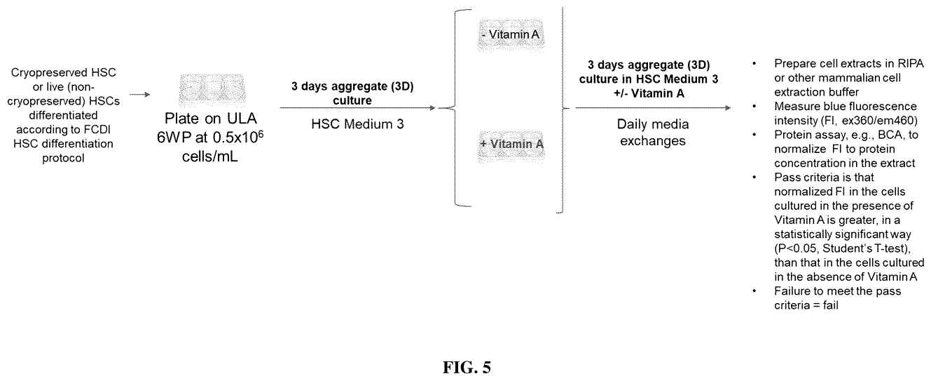

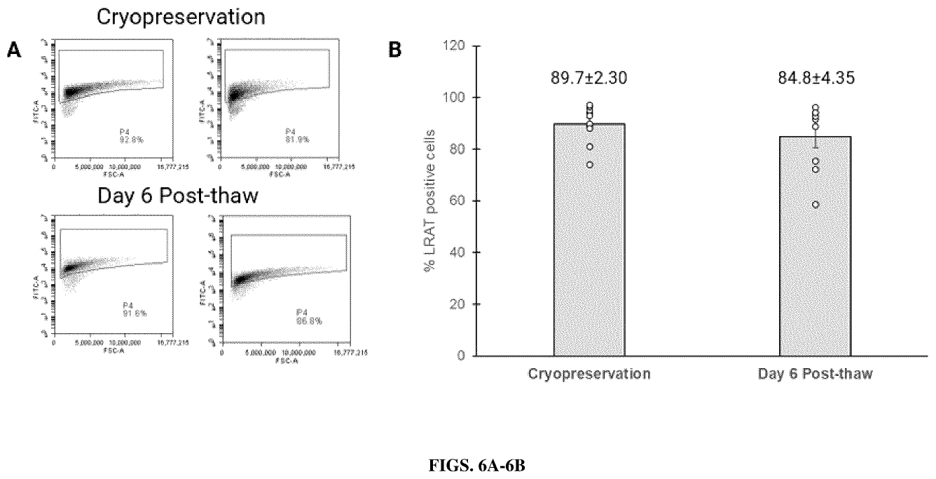

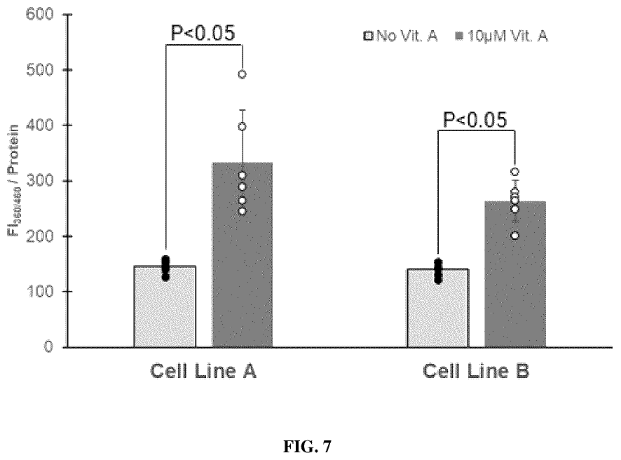

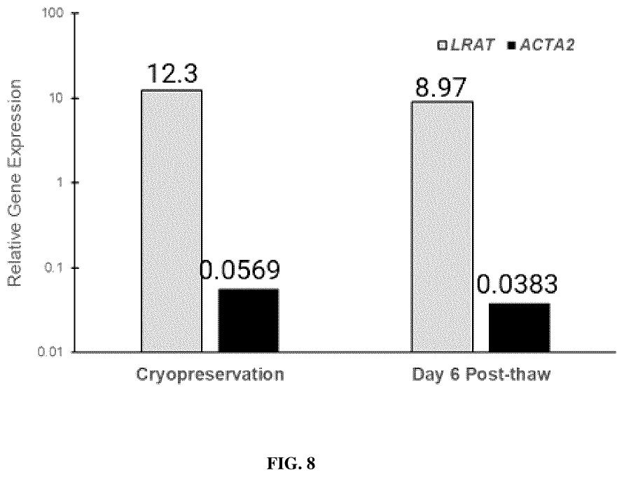

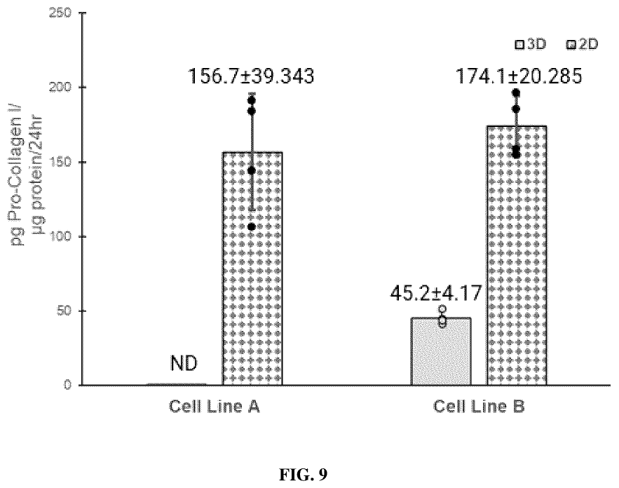

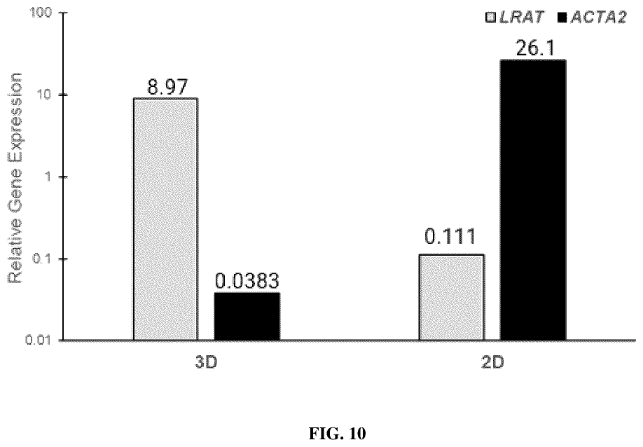

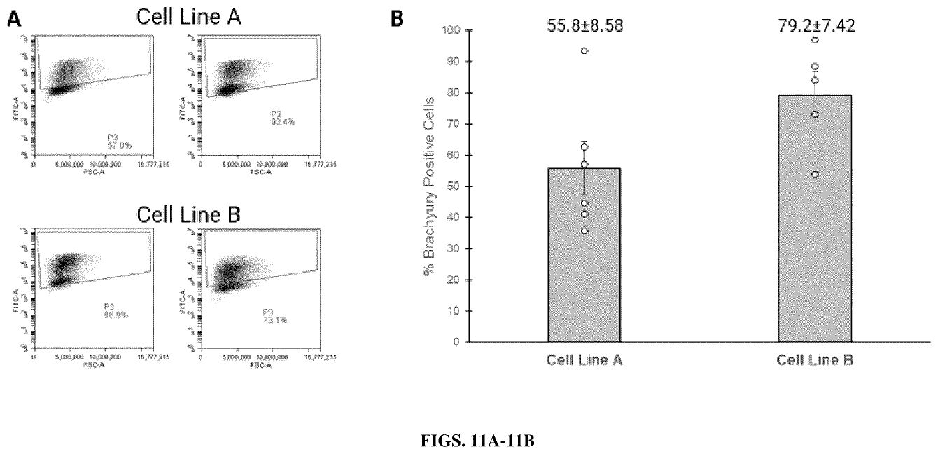

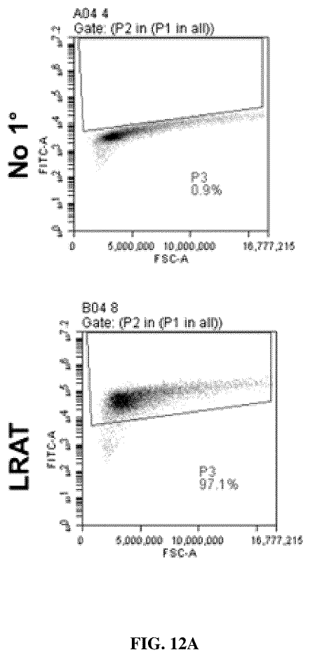

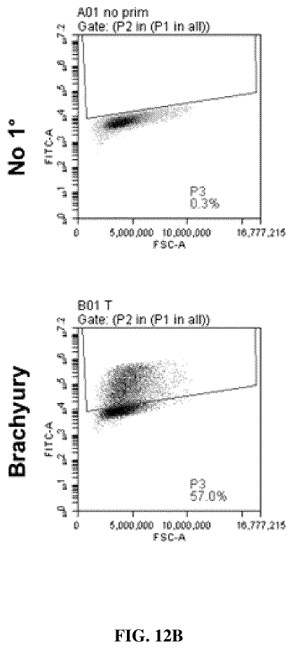

Further, the Vitamin A storage capacity of the HSCs was assessed by a Vitamin A assay (FIGS. 4 and 5). Vitamin A was present in cell extracts from HSCs cultured for as aggregates (top) or in 2D (bottom) in media containing no added Vitamin A, followed by additional 3 days and 10 μM Vitamin A detected by measuring blue fluorescence and normalizing to the total protein level in the extracts.

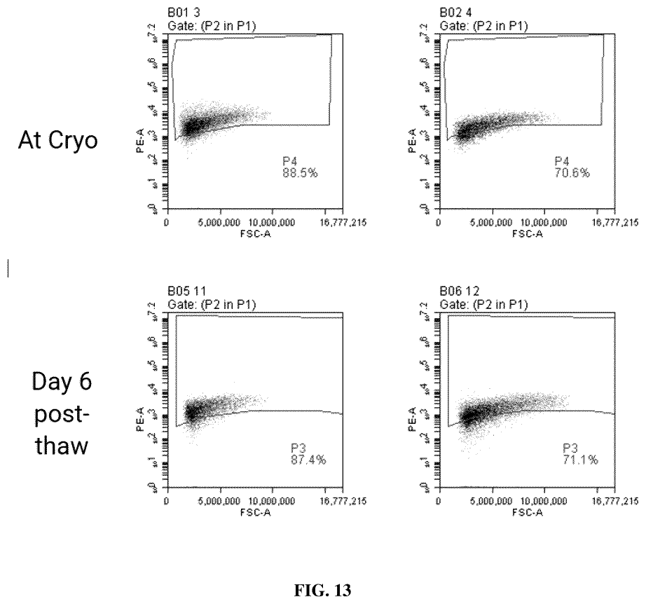

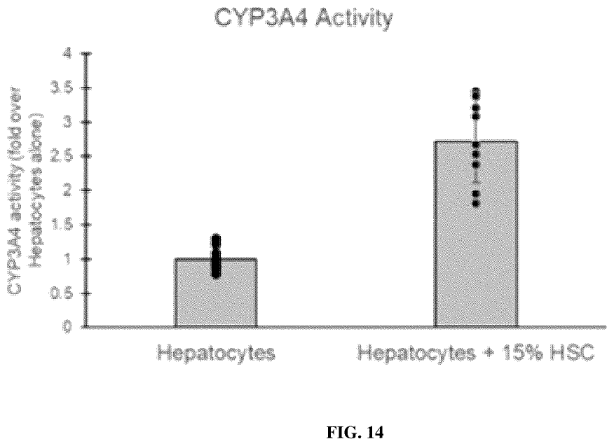

After 5 days of culture, i.e., the time at which the cells were cryopreserved, a sample of the cell aggregates was dissociated, fixed with formaldehyde, and assessed by flow cytometry for expression of pan-HSC, which is expressed in quiescent or activated HSCs. Cryopreserved cells were thawed, and plated at 0.5×10\hat{ }6 cells/mL onto ultra low attachment (ULA) plates in Medium 3 to form aggregates, which were cultured for 6 days after thaw. The cells were then dissociated, fixed with formaldehyde and assessed by flow cytometry in the same way as the cells at cryopreservation. FIG. 13 shows representative flow cytometry plots indicating marker purity measurements.

### Example 2—Methods

Vitamin A storage assay. Live, i.e., not cryopreserved, end of process cell aggregates were distributed onto ultra low attachment (ULA) 24 well plates or cryopreserved cells were thawed and distributed into ULA 24 well plated at the density of ~500,000 cells/mL, and volume of 0.5 ml per well. HSC Medium 3 was used as culture media in both cases. The cells were maintained in HSC Medium 3 until day 3 post plate or post thaw with media exchanges every other day, at which time the media was changed to HSC Medium 3 with or without added Vitamin A (retinol, 10 μM). Cells were cultured in these media for an additional 3 days with daily media exchanges. The aggregates were then transferred to V-bottom 96 well plates, washed twice with DPBS without calcium and magnesium, and lysed with RIPA buffer for 1-2 hours on ice. A portion of each lysate was then transferred to black walled, clear bottom 96 well plates and blue fluorescence intensity (FI, ex. 360 nm/em. 460 nm). Wells containing lysis buffer only were used as assay blanks. After FI measurement, the cell extracts were subjected to BCA protein assay and blank corrected FI was used to normalize to the total protein concentration. A statistically significant difference in normalized FI (with Vitamin A>without Vitamin A, p<0.05 Student's T-test) was considered the passing criteria for the assay.

FACS analysis for LRAT. Cells stained with red fixable live/dead dye and fixed with paraformaldehyde were permeabilized in 2% FBS in DPBS without calcium and magnesium with 1 mg/mL saponin (permeabilization buffer) and stained with anti-human LRAT antibody (Novus/NBP2-50444) diluted in permeabilization buffer overnight at 4° C. Cells incubated in buffer alone (no 1°) were used as negative control. The next day, the cells were washed with permeabilization buffer and incubated with Alexa-Fluor 488 conjugated secondary antibody for ~1 h at room temperature in the dark prior to being analyzed on a flow cytometer. Live cells were gated based on the no 1° negative control.

FACS analysis for Brachyury. Cells stained with red fixable live/dead dye and fixed with paraformaldehyde were permeabilized in 2% FBS in DPBS without calcium and magnesium with 1 mg/mL saponin (permeabilization buffer) and stained with anti-human Brachyury antibody (Abcam/AB209665) diluted in permeabilization buffer overnight at 4° C. Cells incubated in buffer alone (no 1°) were used as negative control. The next day, the cells were washed with permeabilization buffer and incubated with Alexa-Fluor 488 conjugated secondary antibody for ~1 h at room temperature in the dark prior to being analyzed on a flow cytometer. Live cells were gated based on the no 1° negative control.

Human Pro-collagen I alpha ELISA. Live, i.e. not cryopreserved cells cultured on ULA plates and cultured in HSC Medium 3, or cells cryopreserved on day 11, thawed and plated onto ULA plates at 500,000 cells/mL and cultured as aggregates in HSC Medium 3 (quiescent conditions) or same cells plated in HSC Medium 3 onto Matrigel, Collagen, poly-lysine, or other matrix facilitating cell attachment, coated plates at ≥10,000 cells/cm² (activation conditions) were maintained in HSC Medium 3 for 6 days with media exchanges every other day. On day 7, the media was collected and the level of human pro-collagen I alpha secretion into the media was determined using a commercial ELISA kit, e.g., Abcam cat. #ab210966, according to the manufacturer's instructions.

Quantitative PCR (qPCR). RNA was isolated using Quagen RNeasy kit (cat. #74106) and cDNA synthesis was performed using Applied Biosystems High Capacity RNA-to-cDNA Kit (ThermoFisher cat. #4387406) according to the manufacturer's instructions. Gene expression was determined by qPCR using Taqman chemistry with ABI Taqman Gene Expression Master mix (ThermoFisher cat. ##4369542) and ABI Taqman Gene Expression Assay probes (ThermoFisher cat. #4331182) to the manufacturer's instructions. The reactions were performed on Roche480 thermocycler and results analyzed using the instrument software. Expression of the genes of interest (LRAT and ACTA2) was normalized to that of GAPDH. The expression levels of LRAT and ACTA2 are compared before and after cell activation (Under activation conditions includes but is not limited to cultured in 2D and/or in the presence of exogenous TGFbeta.).

Differentiation Protocol of cell population from iPSCs. iPSCs were thawed and plated onto Matrigel coated plate in E8 media with 10 μM Y27632 at ~10,000 cells/well of a 6 well plate (~1,000 cells/cm²). The cells were then cultured for one week in E8 with daily media exchanges in a normoxic (20% O₂/5% CO₂) 37° C. incubator. One week after thaw, media was removed, and the cells were incubated in 0.5 mM EDTA (Ethylenediaminetetraacetic acid) in DPBS (Dulbecco's phosphate buffered saline) without calcium and magnesium for 6-8 minutes, the EDTA solution was removed, the cells were immediately rinsed off the plate with E8 media with 10 μM Y27632, and passaged onto Matrigel coated T150 flasks at 300-500 cells/cm². The cells were cultured for an additional week in a normoxic 37° C. incubator in E8 with daily media exchanges. One week after passaging, the E8 media was replaced with HSC Medium 1 (see below for media compositions); this was considered process day 0. The cells were transferred to a hypoxic (5% O₂/5% CO₂) 37° C. incubator. Cells were cultured in a hypoxic incubator until after day 6 media exchange. The next day (process day 1), HSC Medium 1 was removed and replaced with HSC Medium 2. Cells were maintained in HSC Medium 2 until process day 4 with daily media exchanges. On process day 4, the media was removed, the cells were washed with DPBS without calcium and magnesium, incubated in 1×TrypLE with 10 μM Y27632 (10 mL/T150 flask) at 37° C. for 7-10 minutes, the cells were rinsed off the plate into a conical tube containing HSC Medium 3 in the amount equal to the total amount of 1×TrypLE used, and pelleted at 200×g for 5 minutes. After removing the supernatant, the cells were diluted in HSC Medium 3 to the density of ~500,000 cells/mL and distributed onto ultra-low attachment (ULA) T75 flasks at 20-30 mL/flask. On process day 6, the flasks were placed onto tilted platforms for 10 minutes to settle the aggregates, the media was removed and replaced with equal amount of fresh HSC Medium 3, and the flasks were transferred to a normoxic incubator. The same media exchange procedure was repeated on process day 8. On process day 11, the cells were cryopreserved as follows: cell suspensions were transferred to appropriately sized conical tubes, pelleted at 200×g for 5 minutes, washed with with DPBS without calcium and magnesium, incubated with 1×TrypLE (10 mL for each ULA T75 harvested) for 7-10 minutes, triturated to start breaking up the aggregates. 1×TrypLE was then diluted out with HSC Medium 3, cell suspensions were triturated to complete the aggregate dissociation, the cell suspension was then pelleted at 200×g for 5 minutes, the supernatant was removed, the cells were resuspended in 25 mL of HSC Medium 3, and passed through a 100 μm cell strainer. A sample of the strained suspension was counted, the suspension pelleted at 200×g for 5 minutes, the supernatant was removed, the cells were resuspended in Stem Cellbanker cryopreservative, distributed at 1 mL per cryovial, and cryopreserved in a controlled rate freezer. The cells were cryopreserved at 4-6 million cells per vial, such as 5 million cells per vial.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Amit et al., Dev. Bio., 227:271-278, 2000.
Archer, 2018
Byrne et al., Nature, 450 (7169): 497-502, 2007.
Dunn et al., Drugs 61:1957, 2001.
Fernandes, et al., J. Biotechnology, 132 (2): 227-236, 2007.
International Patent Publication No. PCT/JP2009/062911
International Patent Publication No. PCT/JP2011/069588
International Patent Publication No. WO 02/44343
International Patent Publication No. WO 03/050251
International Patent Publication No. WO 2007/069666
International Patent Publication No. WO2005/123902
International Patent Publication No. WO2020166726A1
International Patent Publication No. WO98/30679
Keller et al., Curr. Opin. Cell Biol., 7:862-869, 1995.
Klimanskaya et al., Lancet., 365: P1636-1641, 2005.
Koui et al., Stem Cell Reports, 16 (12): 3050-3063, 2021.
Ludwig et al., Nat. Biotechnol., 24:185-187, 2006b.
Ludwig et al., Nat. Methods, 3:637-646, 2006a.
Takahashi et al., Cell, 131, 861-872, 2007.
Thomson and Marshall, Curr. Top. Dev. Biol., 38:133-165, 1998.
Thomson and Odorico, Trends Biotechnol., 18 (2): 53-57, 2000.
Thomson et al. Proc. Natl. Acad. Scie. USA, 92:7844-7848, 1995.
U.S. application Ser. No. 12/478,154
U.S. Pat. No. 8,546,140
U.S. Pat. No. 5,843,780
U.S. Pat. No. 6,103,470
U.S. Pat. No. 6,200,806
U.S. Pat. No. 6,280,718
U.S. Pat. No. 6,416,998
U.S. Pat. No. 7,029,913
U.S. Pat. No. 7,442,548
U.S. Pat. No. 7,598,364
U.S. Pat. No. 7,989,425
U.S. Pat. No. 8,058,065
U.S. Pat. No. 8,071,369
U.S. Pat. No. 8,129,187
U.S. Pat. No. 8,183,038
U.S. Pat. No. 8,268,620
U.S. Pat. No. 8,546,140
U.S. Pat. No. 8,691,574
U.S. Pat. No. 8,741,648
U.S. Pat. No. 8,900,871
U.S. Pat. No. 9,175,268
U.S. Patent Publication No. 2003/0211603
U.S. Patent Publication No. 2009/0246875
U.S. Patent Publication No. 2010/0210014
U.S. Patent Publication No. 2011/0104125
U.S. Patent Publication No. 2012/0276636
U.S. Patent Publication No. 2015/0191697
US20070116680
Watanabe et al., Nature Neurosci., 8:288-296, 2005.
Xu et al., Nat. Biotechnol., 19:971-974, 2001.
Yu et al., Science, 318:1917-1920, 2007.

1. An in vitro method for producing quiescent hepatic stellate cells (HSCs) comprising:
(a) culturing pluripotent stem cells (PSCs) in a first media;
(b) further culturing the PSCs in a second media comprising a bone morphogenic protein (BMP) agonist and a glycogen synthase kinase 3 (GKS3) inhibitor, thereby producing mesodermal cells;
(c) culturing the mesodermal cells in the presence of a survival agent, thereby producing aggregates; and
(d) differentiating the aggregates in a third media comprising a transforming growth factor beta (TFGB) inhibitor and a BMP inhibitor, thereby producing a population of quiescent HSCs.

2. The method of claim 1, wherein the first media and second media comprise BMP4.

3. (canceled)

4. The method of claim 1, wherein the second media further comprises basic fibroblast growth factor (bFGF) and/or wherein the third media further comprises vascular endothelial growth factor (VEGF).

5. (canceled)

6. The method of claim 1, wherein steps (a) and (b) are performed under two-dimensional (2D) conditions.

7. The method of claim 1, wherein the BMP agonist is SB4.

8. (canceled)

9. The method of claim 1, wherein the GSK3 inhibitor is selected from the group consisting of CHIR98014, CHIR99021, BIO, and SB-216763.

10. (canceled)

11. (canceled)

12. The method of claim 1, wherein the survival agent is a ROCK inhibitor or a Myosin II-specific inhibitor.

13. The method of claim 12, wherein the ROCK inhibitor is selected from the group consisting of Y27632, chroman, H1152, Fasudil, SR3677, GSK269962, and SB72077B or the Myosin II-specific inhibitor is Blebbistatin.

14.-16. (canceled)

17. The method of claim 1, wherein the TGFβ inhibitor is selected from the group consisting of 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinoxaline (SB525334), 2-(5-Benzo[1,3]dioxol-5-yl-2-ieri-butyl-3H-imidazol-4-yl)-6-methylpyridine hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-benzamide hydrate, left-right determination factor (Lefty), 3-(6-Methyl-2-pyridinyl)-N-phenyl-4-(4-quinolinyl)-1H-pyrazole-1-carbothioamide (A 83-01), 4-[4-(2,3-Dihydro-1,4-benzodioxin-6-yl)-5-(2-pyridinyl)-1H-imidazol-2-yl]benzamide (D 4476), 4-[4-[3-(2-Pyridinyl)-1H-pyrazol-4-yl]-2-pyridinyl]-N-(tetrahydro-2H-pyran-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazole-1-ethanol (R 268712) or 2-(3-(6-Methylpyridine-2-yl)-1H-pyrazol-4-yl)-1,5-naphthyridine (RepSox).

18.-21. (canceled)

22. The method of claim 1, wherein method does not comprise enrichment of the quiescent HSCs and/or mesodermal induction.

23. (canceled)

24. (canceled)

25. The method of claim 1, wherein steps (a) and (b) comprise culturing under hypoxic conditions and/or step (c) comprises culturing under hypoxic conditions followed by culturing under normoxic conditions.

26. (canceled)

27. (canceled)

28. The method of claim 1, wherein the population of quiescent HSCs comprises at least 50% cells positive for lecithin retinol acyltransferase (LRAT).

29. (canceled)

30. (canceled)

31. (canceled)

32. (canceled)

33. The method of claim 1, wherein the method comprises serum-free and/or feeder-free conditions.

34. (canceled)

35. The method of claim 1, wherein the expression of LRAT in cells in the population of quiescent HSCs is greater than the expression of ACTA2.

36. (canceled)

37. (canceled)

38. The method of claim 1, wherein the population of quiescent HSCs express CD271.

39. (canceled)

40. The method of claim 1, wherein the population of quiescent HSCs have the capacity to store Vitamin A, wherein the capacity to store Vitamin A is measured by a Vitamin A storage assay.

41. (canceled)

42. The method of claim 1, further comprising cryopreserving the population of quiescent HSCs as aggregates to maintain quiescence, retain vitamin A storage capacity and retain LRAT expression.

43. (canceled)

44. (canceled)

45. The method of claim 1, further comprising activating the population of quiescent HSCs to produce activated HSCs, wherein activating comprises culturing in two-dimensional culture and/or the presence of exogenous TGFβ.

46. (canceled)

47. The method of claim 45, wherein there is a greater accumulation of Vitamin A in activated HSCs as compared to quiescent HSCs, wherein the activated HSCs secrete collagen.

48. (canceled)

49. (canceled)

50. (canceled)

51. A cell population comprising quiescent hepatic stellate cells (HSCs) from induced pluripotent stem cells, wherein the cell population is generated under serum-free and/or feeder-free conditions, wherein the cell population is produced by the method of claim 1.

52.-63. (canceled)

64. A method of producing a cell population comprising at least 20% LRAT-protein expressing cells, wherein the cell population has not undergone a cell sorting step.

65.-93. (canceled)
